(19) United States
(12) Patent Application Publication
(10) Pub. No.: US 2022/0017937 A1
(43) Pub. Date: Jan. 20, 2022

(54) METHOD FOR DETECTING AND MONITORING THE FORMATION OF BIOFILMS

(71) Applicants: BIOFILM CONTROL, SAINT-BEAUZIRE (FR); UNIVERSITE CLERMONT AUVERGNE, CLERMONT-FERRAND (FR); CENTRE NATIONAL DE LA RECHERCHE SCIENTIFIQUE, PARIS (FR); SIGMA CLERMONT, AUBIERE (FR); INSTITUT NATIONAL DE RECHERCHE POUR L'AGRICULTURE, L'ALIMENTATION ET L'ENVIRONNEMENT ET L 'EVIRONN, Paris (FR)

(72) Inventors: Héloïse BOUDAREL, FIGEAC (FR); Jean-Denis MATHIAS, PLAUZAT (FR); Benoît BLAYSAT, SAINT AMANT TALLENDE (FR); Michel GREDIAC, AUBIERE (FR); Stéphanie BADEL-BERCHOUX, PIGNOLS (FR); Christian PROVOT, LE CENDRE (FR)

(21) Appl. No.: 17/296,654

(22) PCT Filed: Nov. 25, 2019
(86) PCT No.: PCT/EP2019/082345

§ 371 Date: May 25, 2021

(30) Foreign Application Priority Data
Nov. 30, 2018 (EP) 18306593.7

(51) Int. Cl. C12Q 1/02; C12Q 1/18

(52) U.S. Cl. CPC C12Q 1/02; G01N 2015/1075; C12Q 1/18

(57) ABSTRACT

The present invention relates to a method for detecting and/or tracking and/or characterizing the formation of a biofilm. The present invention also relates to a device for detecting and/or characterizing the formation of a biofilm suitable for implementing the method. The present invention can be used in particular in the analytical fields, in biological and enzymological research, in the pharmaceutical field and/or in the medical field.

### TECHNICAL FIELD

The present invention relates to a method for detecting and/or tracking and/or characterizing the formation of a biofilm.

The present invention also relates to a device for detecting and/or characterizing the formation of a biofilm suitable for implementing the aforementioned method.

The present invention can be used in particular in the analytical fields, in biological and enzymological research, in the pharmaceutical field and/or in the medical field.

In the description below, references in square brackets ([ ]) refer to the list of references presented at the end of the text.

### STATE OF THE ART

Biofilm is the main way of life for bacteria since it is the preferred form of life for 90% of bacteria in the natural environment. Unlike the planktonic lifestyle in which bacteria live in suspension, biofilm forms a heterogeneous and complex community of bacteria that attach to a surface and bind to each other through the secretion of an extracellular matrix made up of proteins, sugars and water. The formation of biofilm is a characteristic common to a large number of bacteria. Therefore, it was admitted by the community of microbiologists that the biofilm lifestyle is a full stage in the life of the bacteria. The planktonic form would only be a transition to the biofilm. As with many living species, collectivity is necessary for their survival. This form of community life gives bacteria significant metabolic benefits. The adhesion capacity of biofilms on natural or artificial surfaces is very high and their resistance to all kinds of stress makes them difficult to remove. Biofilms are indeed very resistant to chemical (antibiotics, for example) and mechanical stress (fluid, for example). The increased resistance of biofilms makes them ubiquitous. Although they may be used for innovative processes of bioremediation or synthesis of chemical compounds for example, biofilms may prove to be a real nuisance in various fields where bacterial contamination is to be banned such as in the medical environment, food industry and in certain human environments. In France, biofilms are responsible for 50 to 60% of nosocomial infections (Bryers, 2008) and 40% of food poisoning.

Studies show that by 2050, the number of deaths from a recalcitrant infection could exceed the numbers of deaths from cancer (O'Neill, 2014). In addition, many industries also suffer from the resistance of biofilms. The presence of a thick layer of biofilms on ship hulls increases friction on the water and leads to 35-50% overconsumption of fuel (Schultz et al., 2011). These few figures prove that the study of biofilms represents a considerable socio-economic stake.

In particular, the study of biofilms is essential in the hospital environment, for which the difficulties of biofilm elimination are a public health problem. The resistance of biofilms stems in large part from the mechanical integrity provided by the extracellular matrix. This material provides cohesion between bacteria and adhesion to surfaces. Bacteria protected by extracellular polymeric substances (EPS) in biofilms may survive even after a decontamination procedure and represent a source of infection for humans and animals. In addition, the viscoelastic nature of the matrix allows the biofilm to adapt to shear stresses that the natural environment of the biofilm may induce. The matrix serves as a means of protection for bacteria and allows them to acquire great resistance to their environment.

Also, studies on biofilms allow us to determine how biofilms develop, change and break off. Thus, it is necessary to study their structure and the phenomenon of detachment in order to control their development. Knowledge of the structures of biofilms makes it possible to predict their influences on certain systems. One of the interests of determining the mechanical properties of biofilms is being able to predict and control the formation, accumulation and spread of bacteria that spread infections.

The first observations of biofilms date back to the 17th century but investigations around biofilms remain relatively recent (35 years) with their definition proposed by Costerton et al. (1978). Bacteria have long been studied in their free, isolated and planktonic form. Nevertheless, in a principle of survival, the bacteria attach themselves to a solid support and secrete viscous substances to form a biofilm. Biofilms are made up of bacterial communities that adhere to each other and to a surface through the secretion of a polymer matrix. At the end of the 90s, there was an exponential explosion in the number of publications dealing with biofilms. The biofilm behavior of organisms is increasingly taken into consideration, particularly in the health sector where biofilms develop on a large number of surfaces such as catheters, valves, prostheses, teeth, skin or even mucous membranes, etc.

The formation of a biofilm is a dynamic and non-static process that takes place in several steps detailed below:
reversible adhesion: suspended in a fluid, bacteria approach a surface and adhere to it reversibly through physicochemical attraction forces such as Van der Waals forces. This proximity to a surface is favored by Brownian agitation, gravity or the agitation of the fluid. Some microorganisms are endowed with their own means of propulsion which give them motility. These means of propulsion are made up of protein structures (flagellum, pili) located in the envelopes of the bacteria and allow the bacteria to move independently;
irreversible adhesion: bacteria multiply by cell division and continue to bind to the surface. Furthermore, the modification of the gene expression profile of the adhered bacteria leads to the synthesis of structures on the surface of the bacteria and the secretion of extracellular polysaccharides that irreversibly bind the bacteria to each other and to the surface;
formation of microcolonies: the growth of biofilm organisms and the adhesion of new bacteria to the biofilm formed create microcolonies;
maturation: the microcolonies develop and the biofilm is structured in three dimensions according to the environmental conditions. Channels letting fluids loaded with nutrients circulate emerge through the microcolonies;
detachment: release of specialized free cells or biofilm aggregates that allow colonization of another surface, starting a new cycle (Davies and Marques, 2009). Dispersed bacteria have a predisposition to form microcolonies (Kragh et al., 2016).

A biofilm is mainly made up of microorganisms embedded in an EPS matrix secreted by the microorganisms. Microorganisms represent 10% of the dry mass of the biofilm (Flemming and Wingender, 2010). By analogy, Flemming et al. (2007) see biofilms as cities of microbes living in houses, represented by the extracellular matrix, sheltering them from their physical and chemical environment. Biofilms are highly hydrated since 90-99% of the biofilm is made up of water. The rest of the biofilm is made up of biological macromolecules. These compounds include polysaccharides, proteins, extracellular DNA (eDNA) and RNA. The matrix or the EPS compounds represent 90% of the dry mass. The matrix acts as a binder of bacteria and EPS compounds play an important role in the development and life of the biofilm (Das et al., 2013). Depending on their strain of origin and the growing conditions in terms of nutrients and physical and chemical stress, the rate of extracellular matrix production and its composition are different. Therefore, the proportion of each of the elements contained in a biofilm is determined by a given number of factors, including the type of nutrient, hydrodynamic conditions and temperature.

Observations of sections of biofilms under the microscope show that the composition of the biofilm is highly heterogeneous (Lawrence et al., 1991). The bacteria colonies are traversed by channels through which fluids circulate ensuring the supply of nutrients. The behavior of the biofilm is driven by a very complex and dynamic micro-structure.

Bacteria in biofilms develop properties that planktonic bacteria do not have.

Quorum sensing allows bacteria to communicate and organize to collaborate. This collaboration is also characterized by horizontal gene transfer (Madsen et al., 2012). Bacteria in biofilms are more resistant to biocides than their planktonic analogues. While in the 1990s tolerance to antibiofilm agents was attributed to diffusion problems, it was later found that other phenomena were responsible for tolerance to anti-infective agents. For example, the ability of bacteria to adapt and differentiate allows them to evolve in the direction of their preservation. In general, biofilms are more resistant to cleaning, whether chemical or mechanical. In addition, frequent and repeated exposure to antibiotics may lead to the phenomenon of multidrug-resistant bacteria, meaning that antibiotic treatments no longer have an effect against them. All these characteristics mean that biofilms are present in all environments, that they are resistant and that they become reservoirs of infection by the release of germs. In the food industry, they are present in bioreactors where they are used for the production of alcohol, acids or polysaccharides. In the textile industry, biofilms are studied to make nylon. On the other hand, they serve as an alternative to mechanical finishes on certain denim clothing (sandblasting, etc.). Also, studies on biofilms are increasingly numerous and often at the interface of several disciplines. The study of biofilms from a mechanical point of view is complex due to its multi-scale and multi-physical nature. However, it represents a real scientific issue. The results of mechanical studies on biofilms reported in the literature remain ambiguous. Few of the protocols are standardized and the multiple test models developed by the research teams make it difficult to compare results.

Therefore, there is a real need to find a means and/or method overcoming these deficiencies, drawbacks and obstacles of the prior art, in particular a method allowing a reproducible and comparable study of the mechanics of biofilms.

The search for elements that exclusively characterize biofilms is also of great interest. Among the near or future research topics, we may cite the following issues: locating bacteria within biofilms, quantifying flows, visualizing the interior of biofilms and understanding interactions within multispecies biofilms. The ability of bacteria to form a biofilm is of interest to develop strategies to fight against biofilms or to positively use biofilms, in bioremediation applications for example. The prevention of biofilm formation is a major issue in the health field. Currently, the usual doses of biocidal substances effective in the case of bacterial infections are often ineffective once the biofilm is installed. Therefore, the detection of biofilm formation in vitro is crucial information, especially for the development of prophylactic treatments. Many protocols have been proposed for the analysis of biofilm formation (Azeredo et al., 2017). However, their variability and their number do not allow, in particular, a comparison of the results obtained and/or a uniformity of results.

Therefore, there is a real need to find a means and/or method overcoming these deficiencies, drawbacks and obstacles of the prior art, in particular a standardized method allowing quick and reliable biofilm formation.

There are methods used to try to characterize biofilms. For example, the following methods may be mentioned: microbiological enumeration on solid medium: The enumeration of colony-forming units (CFU) allows a count of microorganisms in a microbiological culture on a solid medium. This protocol is limited to bacteria that grow on an agar plate. This method is also very time consuming. Microtiter plate (Tissue Culture Plates): In this method, no flow circulates and growth takes place in a closed medium. The biomass present in the wells is made visible with the help of dyes. The magnitude of the staining, related to the amount of biofilm, is read from the optical density of the well. The results are classified into three categories: non-biofilm-forming inoculum, moderate inoculum, or strong biofilm-forming inoculum. This method is indirect. Nevertheless, the lack of a standardized protocol and the lack of reproducibility are the notable drawbacks of this method. MBEC™ System (Calgary device): The MBEC™ (Minimal Biofilm Eradication Concentration) system is part of a category of systems allowing the production of reproducible biofilms for the simple and simultaneous evaluation of several biocidal agents at different concentrations. Using microtiter plate-based methods, the Calgary model was developed to avoid bias due to bacterial sedimentation (Ceri et al., 1999; Harrison et al., 2010). The Calgary device consists of a two-part container. The lower part consists of a 96-well micro-plate. The upper part is a cover made up of spikes which coincide with the center of the wells and which fit into each of the wells of the plate. The biofilm is formed on the spikes. The device is used to characterize biofilm formation and assess susceptibility to biocides (Melchior et al., 2007; Arias-Moliz et al., 2010). Dynamic underflow models are methods where the culture medium is open, that is, there is a continuous supply of nutrients. Hydrodynamic flows influence the formation of the biofilm. The type of flow as well as the shear stress generated by the flow has an effect on the adhesive strength of the biofilm. Therefore, these elements are constraints to the reproducibility of the results and also to the reproducibility of biofilms as they are in nature. Robbins apparatus: It consists of a series of coupons inserted into a chamber in which the bacterial medium circulates (Kharazmi et al., 1999) (FIG. 1.3). Coupons may be removed (and replaced) for study without disrupting the flow and in a sterile manner (Coenye et al., 2008). This model was used, in particular, in the context of the study of catheter material (Nickel et al., 1985). However, this process involves destruction of the biofilm and does not allow its mechanistic study. Infusion reactor: This device simulates an environment with low shear stresses. It is used for applications in medical environment (catheter, lung disease, oral-dental biofilm). It only allows the formation and study of biofilms at the air-liquid interface. Microfermenters: Several types of microfermenters are available and sold commercially:

CDC (Center for Disease Control) biofilm reactor (CBR): this reactor consists of a cylindrical container constantly replenished with nutrients, in which plastic tubes are placed. These tubes have locations dedicated to the placement of coupons which will be colonized by the biofilm. The glass container is placed on a magnetic stirrer to create a circular agitation of the fluid. The flow generated by the rotation is adjustable so that it may be laminar or turbulent. This agitation creates a constant shear stress of moderate to high intensity. The coupons are removable once the surface has been colonized by a biofilm. This reactor allows the repeatable formation of biofilm samples from various strains (Goeres et al., 2005);
Rotating disk biofilm reactor: in this system, the coupons are placed on the bottom of the cylindrical glass container. The platform on which the coupons are placed is rotated while the bacterial medium is regenerated by circulation within the container. The shear stress generated by the rotation of the platform is moderate to strong. However, it remains lower than for the CDC;
Rotating annular reactors: in this configuration, the reactor is made up of two concentric cylinders, one of which is rotating. The samples are blades attached to the rotating cylinder.
Flow-cell: The main drawback of the dynamic models presented above is the difficulty of following the formation of the biofilm live. The flow chambers are transparent cells in which the bacterial medium circulates. The material used for the chamber is suitable for visualization by microscopy and allows observation of the development of the biofilm. However, its implementation requires great technicality. The nucleation of the bubbles formed by the fluid constitutes an important problem. Furthermore, observation in a flow chamber is not very suitable for high throughput because relatively few samples are analyzed simultaneously. Microfluidic platform: These highly integrated, or even automated, devices were developed to be able to observe the behavior of bacteria in a controlled environment, both hydrologically and chemically. Small channel systems distribute the growth fluids to the biofilm formation area. The quantities to be analyzed involved in this type of model are very limited. These methods remain little practiced because they are expensive, not very reusable and require a great technicality. As a result, few publications report the use of microfluidic platforms and therefore there is little feedback.

Therefore, there is a real need to find a means and/or method overcoming the deficiencies, drawbacks and obstacles of these methods.

There is also a real need to find a means and/or method overcoming these deficiencies, drawbacks and obstacles of the prior art, in particular a method which may be used regardless of bacteria culture conditions, and which is quick.

The BioFilm Ring Test® (BRT), based on microplate devices, developed by BioFilm Control, makes it possible to evaluate at high yield the ability of microorganisms to form a biofilm. It does not require staining of the sample, nor any washing, thus limiting the deterioration of the biofilm. BRT applications are found in microbiology and the pharmaceutical industry. The test is available in several derivatives corresponding to different applications: high throughput screening of antibacterial agents and preventive and curative antibiotics, characterization of anti-biofilm coating agents, study of the impact of enzymatic activity on the formation of biofilm and degradation. The application of BRT for the screening of antibiotic molecules is called Antibiofilmogramme®. This test results in the determination of the MICb (minimum inhibitory concentration for the biofilm) making it possible to select from among the antibiotics effective on planktonic bacteria (MIC) those which also have a preventive activity on the formation of biofilm. However, this method does not make it possible to determine the kinetics of biofilm formation, for example continuously, without time bias. Also, this method does not allow specific characterization of the mechanics of biofilms. There are inherent links between the metabolism of bacteria living in the form of biofilm and the mechanical properties of the material consisting of the community of bacteria and the matrix. The development of effective strategies to control the presence of biofilms or the development of biotechnological processes based on the use of biofilms require the understanding of the mechanisms inherent in the biofilm lifestyle and the knowledge of the mechanical parameters describing the behavior of the biofilm in its environment. In this context, many test methods have emerged to evaluate these parameters. They are adapted from methods for testing and identifying parameters of conventional engineering materials or inert fluids. However, the lack of standardization, both in the field of mechanical tests and in the methods of identifying mechanical parameters for the characterization of living materials, remains a scientific obstacle. The quest for standardization is problematic since biofilms are living structures. This characteristic leads to complexity in the study of mechanical properties. These are spatially and temporally heterogeneous. Indeed, due to their composition, biofilms are composites. In addition, bacteria have the ability to dynamically modulate mechanical properties in response to the stress they experience. At present, the mechanical properties of biofilms are ambiguous. The parameters reported in the literature are difficult to interpret and compare given the lack of standardization in the various ways in which biofilms are tested. The definition of standardized protocols appears essential to create an interdisciplinary community. The spatio-temporal heterogeneity of biofilms represents a real scientific issue, but the issues related to the living nature of this type of material are real issues. Living materials are fragile samples that should be handled with care. In vitro testing of living samples may lead to rearrangements that bias the results. On the other hand, one should expect a greater variability in the results compared to common engineering materials which are inert. In the case of biofilms, physiological conditions often require non-invasive imaging methods, but precise characterization of sample geometry may be difficult to achieve. Also, the flatness of surfaces often required for field measurements and the control of out-of-plane displacements represent constraints specific to these materials.

Therefore, there is a real need to find a means and/or method overcoming these deficiencies, drawbacks and obstacles of the prior art, in particular a method making it possible to obtain reliable, reproducible, comparable results, and also to reduce costs and the time to obtain said results.

### DESCRIPTION OF THE INVENTION

The present invention makes it possible to solve the problems and drawbacks of the methods of the state of the art by providing a method for detecting and/or tracking and/or characterizing the formation of a biofilm comprising the following steps:

a) carrying out a temporal succession of observations of a solution comprising at least one microorganism and a plurality of particles while the solution is maintained under conditions allowing the development of a biofilm by said at least one micro-organism,

b) detecting the presence of the biofilm and/or a characterization of the kinetics of biofilm formation by a comparative statistical analysis of the displacements of the particles studied during the various observations.

The inventors surprisingly demonstrated that the method of the invention makes it possible to observe the formation of biofilm in real time, without altering the biofilm, and advantageously makes it possible to determine the kinetics of biofilm formation in a continuous manner, without time bias.

The formation of the biofilm is accompanied by the development of a material whose three-dimensional structure may be composed of entanglements of bacteria in a polymer matrix whose mechanical behavior is shown to be viscoelastic.

Advantageously, the method of the invention is a non-destructive method, advantageously making it possible to determine the change in the state of the biofilm over time, in particular by analyzing its material properties.

Advantageously, the method of the invention makes it possible to study and determine the mechanical properties of the extracellular matrix of biofilms.

Advantageously, the inventors demonstrated that the method of the invention advantageously allows, in particular, by incorporating particles and/or microbeads into the culture medium, in particular the bacterial medium, and in particular by recording their displacement, for example with an optical device, e.g., viewed under a microscope, to study and determine the mechanical properties of biofilms, in particular the mechanical properties of the extracellular matrix of biofilms.

Advantageously, the inventors have demonstrated that the results of such a process are dynamic and local, for example by means of the kinematics of the displacement of each of the microbeads/particles observed and/or filmed.

Advantageously, the inventors took advantage of the displacement of the microbeads/particles in the biofilm in order to characterize its development.

In this document, the vectors or displacements are independently designated as n̅ or \vec{n}, n being an example of designation of a vector.

According to the invention, the expression “temporal succession of observations” means at least two observations over time. In particular, it shall be understood that at least two observation steps take place while the solution is maintained under conditions allowing the development of a biofilm by said at least one microorganism. For example, the method may comprise from 1 to 1000 observation steps, for example, from 1 to 100 observation steps during which the solution is maintained under conditions allowing the development of a biofilm by said at least one microorganism.

According to the invention, the observation steps may be performed at regular time intervals. For example, the observation steps may be carried out according to time intervals of between 1 to 10 minutes, 2 to 8 minutes, 3 to 6 minutes, or equal to 5 minutes.

In other words, the temporal succession of observations may be carried out at regular time intervals. For example, the observation steps may be carried out according to time intervals of between 1 to 10 minutes, 2 to 8 minutes, 3 to 6 minutes, or equal to 5 minutes.

According to the invention, the observation may be carried out by any suitable means known to a person skilled in the art. It may, for example, be an optical device, for example a microscope, for example Olympus CKX53 inverted light microscope equipped with a 40× objective, a camera, a scanner, a camera, for example PCO Edge or a Basler Ace camera.

According to the invention, the observation may include at least two image pickups of the solution. This could be, for example, an observation comprising taking at least two images. This may, for example, be an observation comprising a series of image pickups taken, for example from 2 to 100,000 images, from 2 to 50,000 images, for example from 2 to 300 images.

According to the invention, the time interval between two successive images during an observation may be designated as Δt. In the present, the time interval Δt must be >0.

According to the invention, each observation may comprise, for each particle of a set of particles observed during said observation, a determination of a trajectory corresponding to successive displacements made by said particle during said observation.

In other words, according to the invention, an observation Oᵢ may, for example be obtained by determining the trajectories of n particles/beads, along a series of N+1 images, for example N is greater than or equal to 1, for example ranging from 1 to 3000, for example ranging from 1000 to 3000. For example, N may be 1≤N, preferably 1000≤N+1≤3000.

According to the invention, the observation of the solution may be carried out for a given time. For example, the observation may be carried out for a time and/or a duration ranging from 0.01 ms to 15 s, for example from 0.1 ms to 10 seconds.

According to the invention, the observation may be carried out by taking images at any suitable frequency known to a person skilled in the art. This may involve taking images with a frequency of 1 to 1000 images per second, for example 100 to 300 images per second, for example 200 images per second.

According to the invention, the image may be a digital image.

In the present document, by digital image is meant any digital image known to a person skilled in the art. It may be, for example, a digital image formed of a two-dimensional orthogonal grid. The smallest constituent entity of this grid is called a pixel (“picture element”). The digital image may therefore be a matrix of pixels quantified by their coordinates and their gray level. The magnitude of the pixel values may depend on the number of bits on which the image is encoded. If the image is encoded in N bits, a pixel may be quantized by 2N different shades of gray. For example, these may be 16-bit encoded images, which represents a range of 65 536 different gray level values. This sampling of information in space and in grayscale depth defines the dynamics of the image. The linear transposition of a continuous signal of the material observed into a sampled signal constituted by a matrix of pixels may require some precautions. Preferably, in order to be able to apply the DIC, the observed surface may be and remain flat during the observation so as advantageously not to introduce scale differences in the images. Preferably, the lighting may be constant and the dynamics of the image adapted to the desired measurement. The objective of the DIC is to find the measured displacement ū corresponding to the true displacement ūₜ of a surface element between an initial state (t=0) and a deformed state (t). The computation of the displacement u may be carried out in each pixel on a restricted portion of the surface of the material. This portion is commonly referred to by its acronym Rol (“Region of Interest”). A selection of an area of the image where the calculation is made may advantageously make it possible, on the one hand, to overcome the image edge pixels which are liable to disappear during the mechanical test and, on the other hand, set aside areas where mechanical information is not relevant.

According to the invention, the trajectory may be determined by any process and/or method known to a person skilled in the art. It may be, for example, the trajectory of particles, for example all the successive displacements made by a particle, for example during an observation.

According to the invention, trajectory, for example trajectory of a bead, may mean that all successive positions occupied, for example by the center of the bead during a series of images, may be successive, for example. According to the invention, the trajectory may not be in two dimensions, the positions may be given by the Cartesian coordinates, x(t) and y(t), in an orthogonal frame of reference centered on the bead, for example at time t=0.

In other words, according to the invention, trajectory, for example trajectory of a particle, may mean all the successive positions occupied, for example by the center of the particle during a series of images, may be successive, for example. According to the invention, the trajectory may be in at least two dimensions, for example two dimensions or three dimensions. For example, when the trajectory is in two dimensions, the positions may be given by the Cartesian coordinates, x(t) and y(t), in an orthogonal frame of reference, for example centered on the particle.

According to the invention, by observation, for example observation Oᵢ, one may observe and/or mean a set of trajectories of an n particle. For example, an n particle may be observed, n being greater than 1, for example ranging from 1 to 350, for example ranging from 150 to 350. For example, n may be 1≤n, preferably 150≤n≤350 particles, for example present in the sample and/or the solution.

In other words, each observation may comprise a set of n trajectories of n particles, for example greater than 1, for example ranging from 1 to 350, for example ranging from 150 to 350. For example, n may be 1≤n, preferably 150≤n≤350 particles present in the sample and/or the solution.

According to the invention, observation Oᵢ may mean a set of trajectories at a time Tᵢ corresponding to the age of the sample (Tᵢ≤0), for example of n particle, for example n is greater than or equal to 1, for example ranging from 1 to 350, for example ranging from 150 to 350.

According to the invention, motion vector means, for example, a vector of components according to the formula [(x(t+Δt)−x(t), (y(t+Δt)−y(t))], ∀t, 0≤t≤NΔt

According to the invention, the position of the particles, for example during observation, may be determined by any method known to a person skilled in the art. For example, the position of the particles may be determined by identifying any point on the particle, for example by identifying the circumference and/or periphery of said particles or the center of said particles.

According to the invention, the method for detecting and/or tracking and/or characterizing the formation of a biofilm may, for example, comprise the following steps:

a. introducing at least one microorganism into a solution,

b. introducing at least two particles into the solution obtained in (a),

c. observation O1 of the solution obtained in (b) at a time (t1),

d. maintaining the solution obtained in (c) under conditions allowing the development of a biofilm by said at least one microorganism,

e. observation O2 of the solution obtained in (d), at a time (t2),

f. comparing observations O1 and O2,

g. determination of the presence and/or characterization of a biolfilm by comparison of the trajectories of the particles observed during the O1 and O2 observations.

According to the invention, the method for detecting and/or tracking and/or characterizing the formation of a biofilm may, for example, comprise the following steps:

a. introducing at least one microorganism into a solution,

b. introducing at least two particles into the solution obtained in (a),

c. observation O1 of the solution obtained in (b) at a time (T₁),

d. maintaining the solution obtained in (c) under conditions allowing the development of a biofilm by said at least one microorganism,

e. observation O2 of the solution obtained in (d), at a time (T₂),

f. determination of the presence and/or characterization of a biolfilm by comparison of observations O1 and O2,

According to the invention, the detection of particles, in particular on an image, for example a digital image, may be carried out by any suitable method known to a person skilled in the art. It may, for example, be a method for comparing images to at least one reference image (imagev). According to the invention, the reference image (imagev) may be determined by any method known to a person skilled in the art. This may, for example, be a process comprising the following steps:
identifying the center of a particle on an image, for example in an area of interest,
selecting a particle image area (imageb) corresponding to a square centered on said particle, and
obtaining a reference image (imagev) according to the following formula:

$$imagev = \frac{1}{4} [ i m a g e t t {e}_{b} + i m a g {e}_{b}^{T} + i m a g {e}_{b}^{R h} + i m a g {e}_{b}^{R v} ]$$

where imagette_{b} is the raw image of the area around a bead, image_{b}^{T} is the transpose of imageb, image_{b}^{Rh} is the horizontal symmetry of imagette_{b}, image_{b}^{Rv} is the vertical symmetry of imagette_{b}.

According to the invention, the transpose of the imagette_{b} may be carried out by any suitable method known to a person skilled in the art. For example, for A=(a_{i,j}) a matrix of M_{n,p}, the transpose of A, noted A^{T} is the matrix with p rows and n columns of general term b_{k,l} defined by: ∀k, 1≤k≤p, ∀l, 1≤l≤n b_{k,l}=a_{l,k}

According to the invention, horizontal symmetry of the imagetteb may be achieved by any suitable method known to a person skilled in the art. For example, for A=(a_{i,j}) a matrix of M_{n,p}, the horizontal symmetry of A, noted A^{Rh} is the matrix with n rows and p columns of general term b_{k,l} defined by: ∀k, 1≤k≤n, ∀l, 1≤l≤p b_{k,l}=a_{k,p-l+1}

According to the invention, vertical symmetry of the imagetteb may be achieved by any suitable method known to a person skilled in the art. For example, for A=(a_{i,j}) a matrix of M_{n,p}, the horizontal symmetry of A, noted A^{Rv} is the matrix with n rows and p columns of general termb_{k,l} defined by: ∀k, 1≤k≤n, ∀l, 1≤l≤p b_{k,l}=a_{n-k+1,l}

According to the invention, the comparative statistical analysis of the displacements of the particles may be carried out by any suitable method known to a person skilled in the art.

According to the invention, the comparative statistical analysis of the displacements of the particles may include in particular a step of comparing the observations.

According to the invention, the comparison step may be carried out by any suitable image comparison method known to a person skilled in the art. It may, for example, be a method of comparing images, for example for an image in shades of gray, comprising an aggregation of the levels of gray, a distribution of the levels of gray, or, for example, for an image in shades of gray, a method of comparing images comprising an aggregation of the color levels. It may, for example, be an image comparison method, for example a digital image comparison method, for example a digital image in gray levels. For example, the image comparison process, in particular digital (DIC) may, for example, be based on the principle of conservation of gray levels between two images of the same surface at successive instants. For example, the images used for the DIC may correspond to the intact and distorted surfaces of the object of study. They may be represented by 2D functions f and g. The comparison may be based on a comparison of the signal amplitude between the reference image and the image of the distorted state. The functions f and g may represent the gray levels interpolated to the pixel whose position is identified by its coordinates (x, y). The problem to be translated consists in determining ū, the motion field sought at the center of a surface element RoI. During an X transformation, a point x̅ in the reference image f moves in X (x̅) in the distorted image g. For any point with coordinates x̅ belonging to the RoI, the conservation of the gray levels between the two instants may be written:

$$f ( \overline{x} ) = g ( \overline{x} + \overline{u} )$$

Advantageously, for example when there are external disturbances of the gray levels, for example caused by differences in lighting or by sensor noise, between the two images f and g, a distance between f and g may be introduced. The minimization of this distance may then be implemented. For example, the amplitude of the signals over an area, here in 1D, may be compared between the reference image and the image of the distorted state. As it stands, determining the displacement u is a poorly posed problem. It may be regularized, for example, by adding a priori information on the solution of the inverse problem. It may be, for example, a kinematic regularization. For example, a kinematic base to which the displacement belongs is defined. In a practical way, the displacement may be approached by a set of families of simple transformations. The field of displacement ū may be decomposed, a priori, on the basis of N functions of form arbitrarily chosen noted Ψᵢ, where i=1, . . . , N. The i are the functions of forms forming a base describing the motion field

$${\overline{u}}_{i} \approx \overline{u} = {\sum }_{i}^{N} {δ}_{i} {Φ}_{i}$$

where, Ψᵢ is the degree of freedom associated with the form function Ψᵢ.

A spatial regularization, by the choice of the size of the zones, support of the kinematics may also be used. The determination of the optimal coefficients of the approximate transformation may require a minimization of the distance between f and g. This distance may be formalized in the form of a cost function. Various functions may be used as described in Pan et al., 2010. This may be, for example, [symbol] the standard in which the conservation of gray levels is sought by minimizing the square of the difference in gray levels:

This function is non-linear. Assuming these are small displacements, it may be linearized by performing a first order Taylor expansion. The previous equation then becomes:

$${u}^{*} = {argmin}_{u ∈ {ℒ}^{2} ( ROI )} {\int }_{RoI} {[ f ( \overline{x} ) - g ( \overline{x} + \overline{u} ( \overline{x} ) ) ]}^{2} d \overline{x}$$

By injecting ū in its linear combination form into the approximation subspace, this system may be put in matrix form:

$${[ {M}_{ij} ]}^{e} [ {δ}_{i} ] = [ {b}_{i} ]$$

In which the terms M and b are known data which depend on the gradient of the reference image, and the residue between the initial image and the “back-deformed” image.

$${M}_{ij} = {\sum }_{ROI} ( {Φ}_{i} \cdot ∇ f ) ( \overline{x} ) ( {Φ}_{j} \cdot ∇ f ) ( \overline{x} )$$

$${b}_{i} = {\sum }_{ROI} ( f - g ) ( {Φ}_{i} \cdot ∇ f ) ( \overline{x} )$$

“.” corresponds to the Euclidean scalar product between two vectors.
The regularization may be carried out iteratively by updating the displacement increment by a Gauss-Newton algorithm. The displacement increment may be obtained by solving the linearized system equation

$$Δ {δ}_{i t} = {M}^{- 1} b$$

Solving the system makes it possible to find Δδᵢₜ and thus calculate the motion field on the Rol element when the δ is found.

$${δ}_{i t + 1} = {δ}_{i t} + Δ {δ}_{i t + 1}$$

The Gauss-Newton algorithm may operate until the stop criterion is reached, for example when Δδᵤ≤10⁻⁵.

Advantageously, the images may be discretized and therefore known to the nearest pixel. As it is, the calculated displacement takes a value in N2. To achieve sub-pixel precision, an interpolation of gray levels at non-integer coordinates may be performed, for example a bi-cubic or “spline” interpolation.

According to the invention, the method may include, when comparing observations, determining the statistical distribution of the possible trajectory of particles and/or the geometry of the possible trajectory of particles.

According to the invention, the method may include, when comparing observations, independently determining by particle the statistical distribution of the possible trajectory of each of the particles and the geometry of the possible trajectory of each of the particles present.

According to the invention, the method may further comprise a global statistical analysis of the displacements made by the particles observed during each observation and/or a calculation of characteristic times of the formation of the biofilm on the basis of the results of the overall statistical analysis.

In the present, by global statistical analysis is meant any statistical analysis known to a person skilled in the art suitable for a displacement analysis, for example of particles. This may be for example an analysis of the distribution, for example based on the variance, for example the sobol index, an analysis of the variance on the distribution, tests of hypothesis on the type of statistical law representative of the displacement distribution.

According to the invention, the global statistical analysis may comprise the calculation of a value of at least one statistical parameter of a distribution of the displacements carried out respectively by the particles of the plurality of particles and an analysis of the variations as a function of time of the values of said at least one statistical parameter obtained for the succession of observations.

According to the invention, the at least one statistical parameter of a displacement distribution may be any statistical parameter known to a person skilled in the art. It may be, for example, the determination of the standard deviation of the displacement distribution and/or trajectories, of statistical moments. For example, the determination of the standard deviation of the displacement distribution may be established according to the following formula:

$$σ = \sqrt{\frac{1}{m} {\sum }_{i = 1}^{m} {( Pi - \overline{P} )}^{2}}$$

where P is the particle motion vector, m=length (P), and P̅=mean (P).

In the present document, the motion vector of the particles may correspond to the vector connecting a previous position to a new position of the particle, therefore the final position vector minus the initial position vector. In other words, the vector may also be referred to herein as a trajectory.

In the present, the motion vector and/or trajectory of each of the particles may be determined during each of the observations, for example by determining the position of the particles on each of the images obtained during said observation.

According to the invention, the global statistical analysis may comprise the calculation of the mean squared displacement (MSD) according to the following formula: MSD (Δt)=Δr² in which Δr is the radial displacement of the particles and Δt the time interval. In the present, the time interval Δt may correspond to the time interval between two images.

In the present, when the observation includes multiple digital images, the coordinates of the vector may be expressed in pixels.

In other words, according to the invention, the method may include determining the statistical distribution of all particles.

Further, according to the invention, the method may include determining the geometry of the trajectory of all the particles. According to the invention, the determination of the geometry of the trajectory may be carried out by any suitable method known to a person skilled in the art.

Advantageously, the variation in the value of the standard deviation of the displacements and/or trajectories, preferably as a function of time, for example of the observation time Tᵢ, may make it possible to characterize the subsequent biofilm.

Advantageously, the inventors demonstrated that when producing a biofilm, the value of the standard deviation of the distribution of the displacements made by the particles varies over time. In particular, the inventors surprisingly demonstrated that when a biofilm is produced, the value of the standard deviation of the displacement distribution increases over time to a maximum value before decreasing, whereas in absence of biofilm production, once the maximum value of the standard deviation of the displacement distribution is reached, it does not vary over time.

Advantageously, the inventors also surprisingly demonstrated that the variation in the value of the standard deviation of the distribution of the displacements effected by the particles over time during the formation of biofilm may be modeled advantageously, making it possible to determine the time(s) of growth and/or decrease in the value of the standard deviation of the distribution and/or the slope(s) of the curve represented by the variations in the value of the standard deviation of the displacement distributions as a function of time. The inventors also demonstrated that the slope variations of the evolution curve of the standard deviation value of the displacement distribution as a function of time corresponded to variations in the “behavior” of the particles in the medium and/or the solution.

According to the invention, the method may further comprise a statistical analysis of the individual contributions of the particles observed from the displacements performed by each particle observed during each observation and an identification of at least a percentage of particles performing the same type of displacement based on the results of statistical analysis of individual contributions.

According to the invention, the statistical analysis of the contributions of each of the particles and/or of the individual contributions of the observed particles may be carried out by any suitable statistical analysis method known to a person skilled in the art. It may, for example, be a method of statistical analysis as mentioned above.

According to the invention the statistical analysis of the contributions of each of the particles and/or of the individual contributions of the observed particles may be performed for each observation.

According to the invention, the statistical analysis of the individual contributions of the particles may include for each observation:
calculating, for each trajectory followed by a particle observed during said observation, a vector composed of parameter values characteristic of the displacements defining the trajectory concerned;
creating a matrix from the vectors calculated for the particles observed during said observation;
decomposing said matrix by a principal component analysis;
identifying at least one major principal component among the principal components resulting from the decomposition;
generating a diagram in at least one dimension of the projections of the various vectors corresponding to the trajectories of the particles observed on said at least one identified principal component.

According to the invention, the characteristic parameters of the displacements may be at least one characteristic parameter which is selected from the group comprising:

length c1 of the diagonal of the rectangle comprising said trajectory according to the following formula:

$$c 1 = \sqrt{\begin{matrix}{( max ( c u m s u m ( X ) ) - min ( c u m s u m ( X ) ) )}^{2} + \\ {( max ( c u m s u m ( Y ) ) - min ( c u m s u m ( Y ) ) )}^{2}\end{matrix}}$$

where max(A) returns the largest component of vector A, min (A) returns the smallest component of vector A and cumsum(B) returns the cumulative sum of vector B.

the average speed c2 on the trajectory:

$$c 2 = mean ( \sqrt{\frac{{X}^{2} + {Y}^{2}}{Δ t}} )$$

where mean(A) returns the mean of the components of vector A

the standard deviation c3 of the speed distribution of each particle at each point of the trajectory

$$c 3 = std ( \sqrt{\frac{{X}^{2} + {Y}^{2}}{Δ t}} )$$

where std(A) returns the standard deviation of the components of vector A

the standard deviation of the distribution of trajectories along the transverse axis c4 or vertical axis c5:

$$c 4 = std ( X ) \\ c 5 = std ( Y ) \qquad$$

where std(A) returns the standard deviation of the components of vector A

the asymmetric distribution of trajectories along the transverse axis c6 or vertical axis c7:

$$c 6 = skw ( X ) \\ c 7 = skw ( Y ) \qquad$$

where skw(A) returns the asymmetry coefficient of the components of vector A

In the present document, max corresponds to the largest component, std corresponds to the standard deviation, mean corresponds to the mean, skw corresponds to the asymmetry coefficient, cumsum corresponds to the cumulative sum, min corresponds to the smallest component of the vector.

According to the invention, the statistical analysis of the contributions of each of the particles and/or of the individual contributions of the observed particles may comprise the determination of the mean squared displacement (MSD) according to the following formula: <Δr²(Δt)> where Δr is the radial displacement of the particle and Δt the time interval.

According to the invention, the vector composed of values of characteristic parameters of the displacements defining the trajectory concerned may be calculated by any method known to a person skilled in the art. It may be, for example, a method comprising a concatenation of all the values of characteristic parameters.

According to the invention, the constitution of a matrix from calculated vectors may be carried out by any suitable method known to a person skilled in the art. This could be, for example, a principal component analysis.

According to the invention, the principal component analysis may be any principal component analysis known to a person skilled in the art. This may, for example, be an analysis allowing the selection of the components of the values which express the maximum variance.

According to the invention, the identification of at least one major principal component among the principal components resulting from the decomposition may be carried out by any method and/or means known to a person skilled in the art.

According to the invention, the predominant principal component(s) may correspond to the components preferably corresponding to at least 40% of the sets of values, preferably to 60% of the sets of values.

The person skilled in the art, by virtue of his general knowledge, will be able to choose and/or determine the principal components, for example the predominant principal components.

According to the invention, the generation of a diagram in at least one dimension of the projections of the various vectors corresponding to the trajectories of the particles observed on said at least one predominant principal component identified may be carried out by any method known to a person skilled in the art.

According to the invention, the diagram may be a one-dimensional, two-dimensional, three-dimensional or four-dimensional diagram, preferably one-dimensional, two-dimensional or three-dimensional.

According to the invention, the number of dimensions of the diagram may be a function of the number of predominant principal components identified. For example, when three principal components are identified, the diagram may be a two-dimensional diagram, each of the dimensions of the diagram corresponding independently to a principal component, the third principal component being added into the diagram.

According to the invention, the method may comprise, from the diagram generated, a step of identifying a cluster of points in said diagram. According to the invention, the identification of a point cluster in said diagram may be carried out by any method and/or analysis method known to a person skilled in the art. It may, for example, be any suitable clustering method known to a person skilled in the art. This could be, for example, a visual analysis of the distribution of points, a K-mean analysis, a k-Medoides analysis.

According to the invention, the method may include calculating the percentage of particles from a percentage of points belonging to a given cluster. According to the invention, the calculation of the percentage of particles from a percentage of points belonging to a given cluster may be carried out by any suitable method known to a person skilled in the art. A person skilled in the art, thanks to his general knowledge, will know how to choose the method according to the diagram.

Advantageously, the percentage of particles calculated from a percentage of points belonging to a given cluster makes it possible to classify and/or characterize the biofilm formed.

According to the invention, the method may further comprise a step of analyzing an evolution over time of said percentage of particles for the various observations.

Advantageously, the analysis of the evolution over time of said percentage of particles for the various observations makes it possible to determine the dynamics of biofilm formation, from a quantitative and qualitative point of view.

According to the invention, the microorganism may be any microorganism known to a person skilled in the art. It could be, for example, a bacterium or a fungus. They may also be prokaryotic cells, for example any bacterium known to a person skilled in the art, for example bacteria included in the group, without being limited to the latter, consisting of Acetobacter aurantius, Actinobacillus actinomycetemcomitans, Agrobacterium tumefaciens, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus stearothermophilus, Bacillus subtilis, Bacteroides gingivalis, Bacteroides melaninogenicus, Bartonella henselae, Bartonella quintana, Bordetella bronchiseptica, Bordetella pertussis, Borrelia burgdorferi, Branhamella catarrhalis, Brucella abortus, Brucella melitensis, Brucella suis, Burkholderia mallei, Burkholderia pseudomallei Calymmatobacterium granulomatis, Campylobacter coli, Campylobacter jejuni, Campylobacter pylori, Chlamydia pneumoniae, Chlamydia psittaci, Chlamydia trachomatis, Chlamydophila pneumoniae, Chlamydophila psittaci, Clostridium botulinum, Clostridium difficile, Clostridium perfringens, Clostridium tetani, Clostridium welchii, Corynebacterium diphtheriae, Corynebacterium fusiforme, Coxiella burnetii Ehrlichia chaffeensis, Enterococcus avium, Enterococcus durans, Enterococcus faecalis, Enterococcus faecium, Enterococcus galllinarum, Enterococcus maloratus, Escherichia coli Francisella tularensis, Fusobacterium nucleatum Gardnerella vaginalis Haemophilus ducreyi, Haemophilus influenzae, Haemophilus parainjluenzae, Haemophilus pertussis, Haemophilus vaginalis, Helicobacter pylori Klebsiella pneumoniae, Klebseilla rhinoscleromatis-klebsiella oxytoca Lactobacillus acidophilus, Lactobacillus casei, Lactococcus lactis, Legionella pneumophila, Methanobacterium extroquens, Microbacterium multiforme, Micrococcus luteus, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma pneumoniae Neisseria gonorrhoeae, Neisseria meningitidis, Nocardia asteroides Pasteurella multocida, Pasteurella tularensis, Porphyromonas gingivalis, Pseudomonas aeruginosa, Pseudomonas maltophilia, Rhizobium radiobacter, Rickettsia prowazekii, Rickettsia mooseri, Rickettsia psittaci, Rickettsia quintana, Rickettsia rickettsia, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Staphylococcus aureus, Staphylococcus epidermidis, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus minor, Streptococcus mitis, Streptococcus mutans, Streptococcus oxalis, Streptococcus pneumoniae, Streptococcus pyogenes, Streptococcus rattus, Streptococcus salivarius, Streptococcus sanguis, Streptococcus sobrinus, Treponema pallidum, Vibrio cholerae, Vibrio comma, Vibrio parahemolyticus, Vibrio vulnificus, Xanthomonas maltophilia, Yersinia enterocolitica, Yersinia pestis and Yersinia pseudotuberculosis, etc.

It may, for example, be any fungus known to a person skilled in the art, for example pathogenic or non-pathogenic fungi, for example fungi responsible for pathologies, for example in human health or not, environmental fungi, fungi chosen from the group comprising yeasts, for example Candida and/or Cryptococcus, for example yeasts responsible for pathologies, for example in human health, for example candidiasis and/or cryptococcosis, and/or the group comprising molds, for example Aspergillus, for example molds responsible for pathology, for example in human health, for example aspergillosis and/or pulmonary mycosis.

According to the invention, the microorganism may be introduced into the solution at a concentration of 1.10⁶ to 2.10⁸ CFU/ml (CFU: Colony-Forming Unit), for example from 1.10⁷ to 8.10⁷ CFU/ml. For example, in a volume of 200 μl, the quantity of microorganism may be between 2.10⁵ and 4.10⁷ CFU, for example from 2.10⁶ to 1.6.10⁷ CFU.

According to the invention, the solution may be contained in a container and/or present on a surface. It may, for example, be any suitable container known to a person skilled in the art. This could be, for example, a culture container.

According to the invention, the term “the surface” means any cultivation surface known to a person skilled in the art. It may be, for example, a glass slide, in Plexiglas or other suitable material. This could be, for example, a glass slide to which a drop of medium and bacteria may be added.

According to the invention, the term “culture container” means any culture container known to a person skilled in the art. This may be, for example, a culture reactor, cups, tubes or wells, for example in microdilution plates.

According to the invention, the culture container may, for example, be enclosure with a closed end, a tube-type, a well, etc. or an enclosure having two openings. It may also be a container comprising a closed end so as to form a flat bottom, a container with a closed end so as to form a hemispherical bottom, a container comprising two open ends. When the container has two open ends, said container may be configured so as to allow the culture medium to flow in a constant flow or in a discontinuous flow.

It may, for example, be a microdilution plate, such as the type of plate defined, for example, by the American National Standards Institute and the Society for Biomolecular Screening (“microplates”) carrying 96 wells, 384 and even 1536, or, conversely, 48 or 24 wells or any other number of wells.

It may be, for example, a culture container made of any suitable material known to a person skilled in the art. It may be, for example, plastic, for example polycarbonate, polypropylene, polystyrene, etc., glass, metal.

These may be, for example, polycarbonate or polypropylene microplates with a flat, conical or round bottom.

It may, for example, be a polystyrene container, for example any polystyrene known to a person skilled in the art.

According to the invention, the solution may also be on a surface. It may be any surface known to a person skilled in the art on which a microorganism may be incubated and/or may develop and/or on which a biofilm may form. It may be, for example, a biotic or abiotic surface. It may, for example, be a surface of a device and/or medical supports, for example dental implants, catheters, prostheses and/or any medical device known to a person skilled in the art on which microorganisms may develop.

In the present document, the term biotic surface means any biotic surface known to a person skilled in the art.

In the present document, the term abiotic surface means any abiotic surface known to a person skilled in the art. It may also be any device surface known to a person skilled in the art on which a bacterium is capable of forming a biofilm. It may for example be the surface of a medical device, for example a catheter, a needle, an implantable chamber catheter (or Port-a-Cath), a valve, for example a heart valve, a prosthesis, for example a joint prosthesis, ligament prosthesis, dental implant, urinary tract prosthesis, peritoneal membrane, peritoneal dialysis catheters, synthetic vascular grafts, stents, internal fixation devices, percutaneous sutures and/or tracheal, ventilation tubes.

According to the invention, the solution may be any solution and/or medium known to a person skilled in the art suitable for the culture of microorganisms.

According to the invention, the solution may be any suitable solution known to a person skilled in the art in which a microorganism may be incubated and/or may grow and/or on which a biofilm may form. It may, for example, be a culture medium, for example any culture medium known to a person skilled in the art and/or commercially available in which at least one microorganism is capable of developing. It may be, for example, a natural or synthetic medium. It may for example be a culture medium for bacterial growth, for example BHI medium (Brain Heart Infusion), LB medium (Lysogeny broth, also called “Luria Bertani”), MH medium (Mueller-Hinton medium), glucose broth, yeast culture medium, for example, Sabouraud medium. A person skilled in the art, based on his general knowledge, will know how to choose the suitable culture medium according to the microorganism.

According to the invention, plurality of particles means at least two particles. According to the invention, said at least two particles may be any particle making it possible to implement the present invention. Advantageously, said particle may be a particle of any shape suitable for the implementation of the present invention, for example in the shape of a bead, a puck, of asymmetric geometric shape, for example with a flat face, etc.

According to the invention, the shape of the particles may be the same or different. Advantageously, the shape of the particles is identical.

According to the invention, the particle may be of any suitable material known to a person skilled in the art. It may be a metal or plastic particle, for example. Any suitable particle size may be used. The size may be chosen, for example, based on the size of the container of the solution and/or of the microorganism. For example, the size of the particles may be less than one tenth of the size of the container, preferably less than one hundredth, even more preferably less than one thousandth of the size of the container. For example, the particles may have a size of, for example, 10 nm to 100 nm, 0.1 to 100 μm.

According to the invention, the method of the invention may be implemented with at least 2 particles, with, for example, from 2 to 10,000,000, from 1,000 to 1,000,000, from 10,000 to 1,000,000, from 100 000 to 1,000,000, from 10,000 to 100,000 particles.

The plurality of particles advantageously makes it possible to observe a plurality of trajectories of said particles. According to the invention, when the method is carried out with a plurality of particles, said particles may have the same size or different sizes. Advantageously, the size of the particles is identical.

When the particles are of different sizes, the small particles may have a size, for example, from 10 nm to 1 μm, for example, from 100 to 500 nm, and the large particles may have a size, for example, from 1 μm to 100 μm, for example from 1 μm to 10 μm, for example from 1 μm to 5 μm.

According to the invention, it is also possible to use a plurality of particles of identical sizes.

According to the invention, the particles may generate a detectable signal. The detection of this signal will depend on the properties of the particle. For example, said at least one particle may be fluorescent, phosphorescent, chemiluminescent, reflective or colored.

For example, in the case where said at least one particle is fluorescent, the fluorescence emitted by the particle may be detected, for example visually, and/or by any optical means known to a person skilled in the art. Said at least one particle may, for example, be illuminated, in order to follow its displacement by means of a light source, for example by a laser beam.

For example, in the case where the at least one particle is phosphorescent, this particle may be visualized, for example visually, by any optical means known to a person skilled in the art.

For example, in the case where the at least one particle is chemiluminescent, the detection of the particle may be formed by adding to the medium the chemical reagent allowing the emission of light energy by the particles.

Advantageously, said at least one particle may be, for example, illuminated, to follow its displacement by means of a light source, for example by a laser beam.

A person skilled in the art will easily understand that for carrying out the present invention, the choice of the visual properties of said at least two particles may also be made according to the solution.

According to the invention, the method may comprise, beforehand and/or during the step of maintaining the solution obtained in (c) under conditions allowing the development of a biofilm by said at least one microorganism, the introduction of at least one compound selected from an antibiotic, an antifungal and/or mixture thereof.

According to the invention, the term compound means any compound, for example natural or obtained by synthesis, chemical semi-synthesis, or obtained by extraction, known to a person skilled in the art capable of modifying the growth/development of microorganisms and/or capable of killing a microorganism. It may, for example, be a biocide, for example a disinfectant, a protection product, a product for combating so-called harmful species and the like and/or any biocide included in the list according to Directive 98/8/EC or Regulation (EU) No. 528/2012, an antibiotic or an anti-fungal.

It may, for example, be an antibiotic chosen from the group comprising fusidic acid, aminoglycosides, beta-lactams, penicillins, beta-lactamase inhibitors, cephalosporins, carbapenems, monobactams, cycloserine, daptomycin, fosfomycin, lincosamides, macrolides and ketolides, mupirocin, nitro-imidazoles, nitrofurans, oligosaccharides, oxazolidinones, polypeptides: bacitracin, glycopeptides, polymyxins and colistin, quinolones, rifamycins, sulfonamides and diaminopyridines, synergistins, tetracyclines, antifungals, or any mixture thereof.

It may, for example, be an anti-fungal chosen from the group comprising Miconazole, Ketoconazole, Clotrimazole, Econazole, Bifonazole, Butoconazole, Fenticonazole, Isoconazole, Oxiconazole, Sertaconazole, Sulconazole, Thiabendazole, Tioconazole, Fluconazole, Itraconazole, Isavuconazole, Ravuconazole, Posaconazole, Voriconazole, Terbinafine, Amorolfine, Naftifine, Butenafine, Mycosubtiline or any mixture thereof.

It may also be any new compound for which it is sought to demonstrate a possible antibiotic and/or antifungal activity, alone or in combination with one or more other compounds.

According to the invention, the antibiotic and/or the anti-fungal may be in solution in the solution, for example in the culture medium, or in dehydrated or lyophilized form, and/or fixed on the surface immersed in said medium.

The attachment means that may be used for the antibiotic and/or the anti-fungal may be any means known to a person skilled in the art for the attachment of an antibiotic and/or an anti-fungal to a surface. It may be, for example, commercially available means or a device, for example, microplate type supports, for example means or a device marketed by NUNC (Denmark), Corning (United States), and Greiner Bio-One (Austria).

According to the invention, one or more compounds, for example one or more antibiotics, may be introduced into the culture medium.

Advantageously, when the method according to the invention makes it possible to test different antibiotics and/or anti-fungal agents alone and/or to search for a combination or association effect of antibiotics and/or anti-fungal agents in a commonly used format called a checkerboard. The implementation of the process of the invention with a mixture of antibiotics and/or anti-fungal agent (combination, association) may advantageously make it possible to detect any additive, synergistic and/or antagonistic effects.

By “additive effect” is meant the addition of the actions of at least two antibiotics making it possible, for example, to find a combination of antibiotics having the same antibiotic effect as an antibiotic alone, but with lower concentrations.

By “synergistic effect” is meant an improvement in the efficacy of an antibiotic and/or anti-fungal by adding at least a second antibiotic and/or anti-fungal, this mixture of antibiotics and/or anti-fungal having an effect greater than the addition of the effect of each antibiotic and/or anti-fungal alone.

Finally, by “antagonistic effect” is meant the inhibition of the action of an antibiotic and/or an anti-fungal on the microorganism by adding at least a second antibiotic and/or anti-fungal. This embodiment may make it possible to select relevant combinations or associations of antibiotics and/or anti-fungal agents (“co-drug”) in order to, for example, inhibit the development of the microorganism.

According to the invention, the antibiotics and/or anti-fungal agents may also be introduced successively into the medium in order to identify additive, synergistic or antagonistic effects, as described above.

Advantageously, the method according to the invention makes it possible to determine/detect and/or identify the sensitivity of microorganisms to compounds. Also, the method according to the invention advantageously makes it possible to identify a new biocide, for example a new antibiotic and/or antifungal. In addition, the method according to the invention may advantageously make it possible to determine the specificity of a compound, for example whether it is an antibiotic and/or an antifungal.

The present invention also relates to a device for detecting and/or characterizing the formation of a biofilm capable of implementing the aforementioned method.

It may be any suitable device known to a person skilled in the art. It may, for example, be a device comprising detection means, for example optical detection means, for example a camera, an electron microscope, analysis means, for example a calculation module, graphic representation means, for example a visual representation module.

Other advantages may also appear to a person skilled in the art upon reading the examples below, illustrated by the appended figures, given by way of illustration.

### BRIEF DESCRIPTION OF THE FIGURES

FIG. 1 shows an example of a digital image comparison. In the diagrams, the abscissa shows the pixels and the ordinate the gray level.

FIG. 2 shows an experimental design of a study of biofilm formation through microbeads trapped in the bacterial medium.

FIG. 3 shows diagrams representing the relative BioFilm indices (rBFI) (ordinate) as a function of incubation time (abscissa) for P. aeruginosa (crosses or vertical lines), S. aureus (crosses or circles) and without bacteria (cross or triangle). The shaded areas on the left graph represent the standard deviation for the two replicas.

FIG. 4 shows diagrams illustrating the different bead speeds for four different types of displacement encountered during biofilm growth, namely passive displacement (a), ejected/dredged displacement (b), slowed displacement (c) or displacement of linked beads (d).

FIG. 5 shows the mean square displacement (MSD) (ordinate) as a function of time in seconds (abscissa) for beads taken in a suspension of P. aeruginosa for different incubation times: 0.45, 75 or 105 minutes.

FIG. 6 shows the mean square displacement (ordinate) as a function of time in seconds (abscissa) for beads taken in a suspension of S. aureus for different incubation times: 0, 40, 120 or 160 minutes.

FIG. 7 is a diagram showing the evolution of the standard deviation of displacements of all particles (ordinate) as a function of the incubation time in minutes (abscissa) of S. aureus or not.

FIG. 8 shows two diagrams representing two models of the evolution of the standard deviation of particle displacements as a function of incubation time. In the figure, a and b respectively represent the slope of the first linear part and the vertical intercept, σ1 a first break in the slope and σ2 a second break in the slope. In the diagrams, the ordinate corresponds to the standard deviation of particle displacements and the abscissa to the incubation time.

FIG. 9 shows the results obtained after principal component analysis after incubation with bacteria; FIG. 9(a) shows a bar diagram representing the analysis result in principal component of the displacement parameters or trajectories of the particles; FIG. 9 (b) shows a graph as a function of the first (PCA1) (abscissa) and second principal components (PCA2) (ordinate). FIG. 9(c) shows the proportion of particles/beads as a function of the dot distribution based on time in FIG. 9(b). The light gray dots correspond to particles/beads with blocked displacements, the ordinate corresponds to the percentage of beads and the abscissa to the incubation time in minutes.

FIG. 10 shows the results obtained after principal component analysis after incubation without bacteria; FIG. 10(a) shows a histogram representing the analysis result in principal component of the displacement parameters or trajectories of the particles; FIG. 10(b) shows a graph according to the first and second principal components; FIG. 10(c) shows the proportion of particles/beads as a function of the dot distribution based on time in FIG. 10(b). The light gray dots correspond to particles/beads with blocked displacements, the ordinate corresponds to the percentage of beads and the abscissa to the incubation time in minutes.

FIG. 11 shows the results obtained after principal component analysis after incubation in the presence of S. aureus; FIG. 11(a) shows a bar diagram representing the analysis result in principal component of the displacement parameters or trajectories of the particles; FIG. 11(b) shows a graph according to the first and second principal components. FIG. 11(c) shows the proportion of particles/beads as a function of the dot distribution based on time in FIG. 11(b). The light gray dots correspond to particles/beads with blocked displacements.

FIG. 12 corresponds to a diagram representing the evolution of the standard deviation (ordinate) as a function of incubation time for P. aeruginosa. The slope break occurs at 34 minutes.

FIG. 13 shows the results obtained after principal component analysis after incubation with P. aeruginosa; FIG. 13(a) shows a histogram representing the analysis result in principal component of the displacement parameters or trajectories of the particles, the ordinate shows the percentage of variance, the abscissa the components; FIG. 13(b) is a graph according to the first (abscissa) and second principal components (ordinate); FIG. 13(c) shows the proportion of particles/beads as a function of the dot distribution based on time in FIG. 13(b). The light gray dots correspond to particles/beads with blocked displacements.

FIG. 14 shows two diagrams (A and B) representing the evolution of the standard deviation of displacements of all the particles (ordinate) as a function of the incubation time in minutes (abscissa) of the medium alone (blank), S. aureus alone, S. aureus with Vancomycin, or S. aureus and fusidic acid. The indicators on the diagrams indicate the corresponding curves.

FIG. 15 shows the results obtained after principal component analysis after incubation of S. aureus with Vancomycin; FIG. 15(a) shows a bar diagram representing the analysis result in principal component of the displacement parameters or trajectories of the particles, the ordinate shows the percentage of variance, the abscissa the components; FIG. 15(b) shows a graph according to the first (abscissa) and second principal components (ordinate); FIG. 15(c) shows the proportion of particles/beads as a function of the dot distribution based on time in FIG. 15(b). Light gray dots correspond to particles/beads with blocked displacements.

FIG. 16 shows the results obtained after principal component analysis after incubation of S. aureus with Vancomycin; FIG. 16(a) shows a bar diagram representing the analysis result in principal component of the displacement parameters or trajectories of the particles, the ordinate shows the percentage of variance, the abscissa the components; FIG. 16(b) shows a graph according to the first (abscissa) and second principal components (ordinate); FIG. 16(c) shows the proportion of particles/beads as a function of the dot distribution based on time in FIG. 16(b). The light gray dots correspond to particles/beads with blocked displacements.

FIG. 17 shows diagrams representing the relative BioFilm indices (rBFI) (ordinate) as a function of incubation time (abscissa) for S. aureus alone, with Vancomycin, with fusidic acid and without bacteria (blank). The indicators in the figure indicate the corresponding curves.

FIG. 18 shows diagrams representing the number of colonies formed per well (CFU/well) (ordinate) as a function of incubation time (abscissa) for S. aureus alone, with Vancomycin, with fusidic acid. The indicators in the figure indicate the corresponding curves.

FIG. 19 shows diagrams of the mean square displacement (ordinate) as a function of time in seconds (abscissa).

FIG. 20 shows two images of a particle; the left image corresponds to the raw image and the right image to the image after transformation.

FIG. 21 shows an image of a well comprising a medium with bacteria and particles.

FIG. 22 shows an example of an area of interest (particle) used to track and/or calculate particle displacements, in particular by image correlation.

FIG. 23 is a grayscale image of a well illustrating the background noise of the sensor.

FIG. 24 is a diagram showing a scatter plot representing the variance as a function of the average of the gray level distribution of one pixel over 1000 images.

FIG. 25 represents diagrams of the mean square displacement (ordinate) as a function of time in seconds (abscissa) for the trajectories of 19 beads diffusing in a 68% glycerol solution. The slopes of the graph curves on the right were approximately equal to 1 (1.5±0.1)

FIG. 26 shows diagrams of the mean square displacement (ordinate) as a function of time in seconds (abscissa) of the trajectories of 39 beads diffusing in a BHI solution. The curve slopes of the graph on the right are approximately equal to 1 (0.98±0.11)

FIG. 27 represents a photograph (FIG. 27a)) of a well of a bead trajectory diffusing in the liquid N350 with the presence of a bias due to a flow of the medium and a diagram of the mean square displacement (ordinate) as a function of time in seconds (abscissa) (FIG. 27b)) of seven trajectories of beads present in the same medium, the second-degree quadratic polynomial tendency indicating a flow in the medium.

FIG. 28 shows diagrams representing the displacement of a particle (ordinate) as a function of the frequency of frames per second (fps) during an observation (abscissa).

FIG. 29 shows histograms representing the displacement of a particle (ordinate) as a function of the frequency of frames per second (abscissa), namely for 1000 fps (line 1), 500 fps (line 2) and 200 fps (line 3), during the same observation.

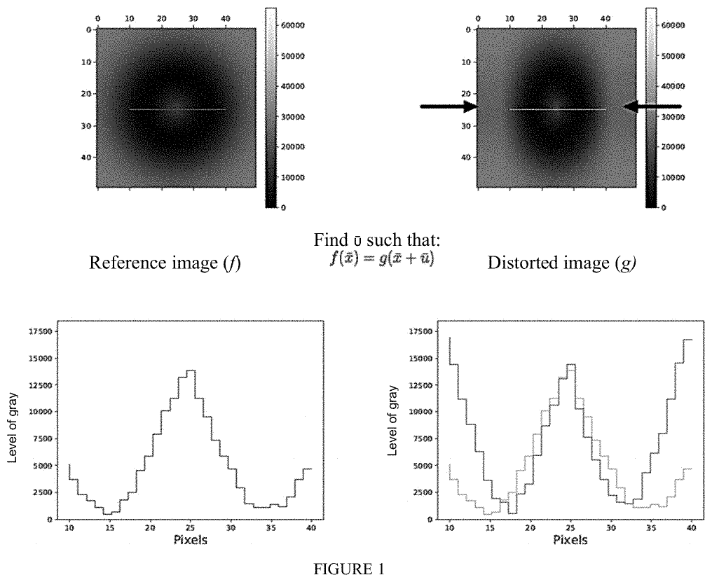

### EXAMPLES

### Example 1. Tracking of Microbeads In Situ for the Detection of Biofilm Formation

In the example below, the particles are independently referred to as beads, particles or microparticles.

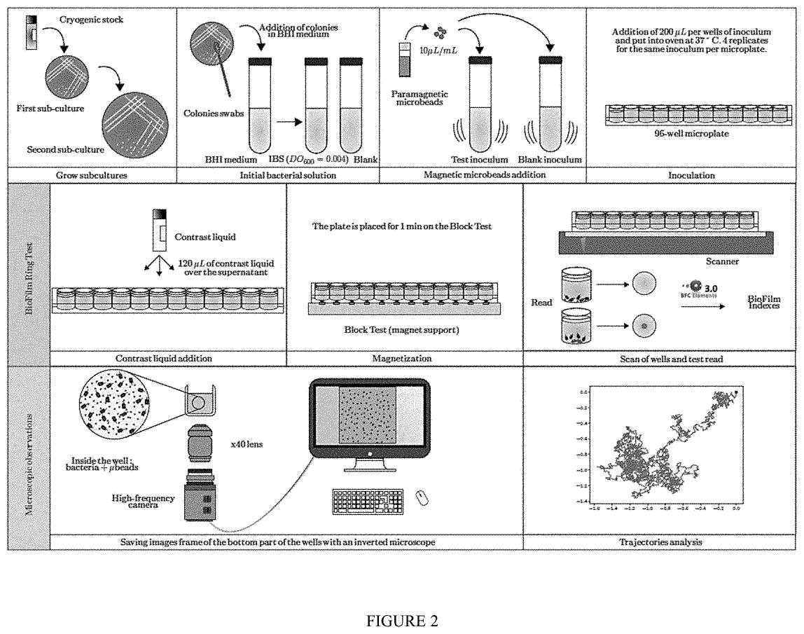

The microbead tracking method presented here is applied to the study of P. aeruginosa and S. aureus biofilms. Biofilm growth kinetics were performed for its strains. Series of microscopic images were extracted at successive stages of biofilm growth. Therefore, the experiments focused on the early stages of biofilm formation studied, from characterizing the BHI medium containing planktonic bacteria and microbeads, to determining the first signs of biofilm formation through the displacements of the microbeads. In the example below, changes in the dynamics of the microbeads were investigated rather than calculating mechanical parameters based on the theory of pure Brownian displacements. Indeed, it is advantageous to be able to distinguish the different displacements of the beads on a criterion other than that of the comparison of MSD (mean squared displacement) which is valid in homogeneous and inert media. Therefore, the objective is to study the formation of biofilms from a mechanical point of view, by determining a revealing parameter of the genesis of a biofilm. By taking advantage of the observation of the displacements of microbeads introduced into the bacterial medium, changes in the trajectory profile were determined and are linked to the activity of sessile bacteria (adhesion or formation of extracellular material). The experimental laboratory was in containment class P2 (“Concerns non-genetically modified class 2 microorganisms which may cause diseases in humans but whose dissemination in the environment is unlikely, which are without risk for community and against which effective prophylaxis or treatments are known”). An Olympus CX53 optical reference microscope was used and the manipulation was carried out under a microbiological safety cabinet. The dynamics of the microbeads within the bacterial liquid over time were observed. The control consisted of a solution of microbeads in the BHI used for the tests. The free displacement of the microbeads in the bacterial medium was visualized at room temperature (20° C.) in transmitted light. From image series recordings of observations of beads moving freely (without external stress) in the bacterial medium, the objective of the analyzes was to detect changes in dynamics in the bead trajectories over time. To that end, series of images at a frequency of 200 images per second were taken at successive times during the growth of biofilm of P. aeruginosa and S. aureus in BHI and in the presence of microbeads, preferably paramagnetic, of diameter 1 μm. The portion of the bottom of the well observed varies between 600 μm² and 1000 μm². FIG. 2 represents an experimental diagram of the steps implemented. A few dozen to a few hundred microbeads could be viewed and tracked in the same series of images. Series of bottom well images were recorded until all of the beads were immobilized within the biofilm, or adhered bacteria. The blocking/immobilization time for most of the beads was strain dependent. A BRT test, in the same culture medium, was carried out in parallel for the strain studied, thus corresponding to a reference in terms of biofilm formation. The results on the analyzes of the displacements of the beads extracted by DIC are presented below.

### 1. Materials and Methods

The materials and methods used were as follows:
a) The protocol for the kinetics of biofilm formation without antibiotic was as follows and was based on observation, until the immobilization of the beads (a few hours), every 5 or 10 minutes, the bottom of a test well and a control well.
The material used was as follows: 1 dish of bacteria subcultured twice; TON4 (paramagnetic microbeads); BHI (Brain Heart Infusion) medium; as many 96-well microplates as incubation times read.
The protocol included the following steps:
1. Preparing 2 mixtures:

Mixture for BRT test: a bacterial solution at OD (600 nm) 0.004 and 10 μl/ml of TON4;

Mixture for control BRT: a BHI+TON4 mixture at 10 μl/ml.

2. Filling 4 wells with each of the mixtures in each of the plates (200 μl/well) according to a plate plan established beforehand;
3. Placing all but one plate in the oven.
4. With the remaining plate, taking a series of images for the test well and the control well;
5. Adding contrast liquid to the wells and performing a BRT (image+BFI analysis);
6. Repeating the protocol from step 4 for all incubation times, removing a plate from the oven at the desired time.
Analysis: The trajectories of all the beads present on the series of images for the test well and the control well were followed, for each incubation time. The evolution of the tendency of the free displacements of the beads over time is sought. The results obtained with the BRT were used as a reference.
b) The protocol for the kinetics of biofilm formation in the presence of antibiotic was as follows and was based on observation, until the immobilization of the beads (a few hours), every 5 or 10 minutes, the background of a test well and a control well. The material used was as follows: 1 dish of bacteria subcultured twice; solution of 1 μm beads (TON4); BHI medium; antibiotics in solution: Vancomycin at 4 μg/mL and fusidic acid at 2 μg/mL (final concentrations in the wells); as many 96-well microplates as observed incubation times.
The protocol included the following steps:
Preparing the antibiotic plates: in the plates, the antibiotics were added by depositing 20 μl. As this deposit was diluted by adding the bacterial suspension (180 μl/well), the solutions deposited had to be 10 times more concentrated than the desired value, i.e., 40 μg/mL for Vancomycin and 20 μg/mL for fusidic acid. The deposition solutions were prepared from existing solutions prepared according to ISO 20776-1: 2006. The solutions were then deposited in the microplates according to an established plate plan/distribution, at a rate of 20 μl/well. The water for injection (WFI) is placed in the control wells.

### Plate Inoculation

1. Preparing a “control” mixture with BHI and TON4 at a rate of 10 μl/mL;
2. Preparing a “strain” mixture with BHI and TON4 at a rate of 10 μl/mL;
3. From the second subculture of the strain, collecting a few colonies with an oese and resuspending them in a tube with 3 mL of BHI;
4. Measuring the OD at 600 nm (OD600 nm) of the suspension and calculating the volume of inoculum to add to the “strain” mixture in order to have an OD600 nm=1/225=0.004444 (the OD600 nm in a well will be 0.004). Inoculum calculation=0.004444 *StrainmixV/OD600 nm
5. Inoculating the “strain” mixture with the strain.
6. After homogenization of the “control” and “strain” mixtures, distributing them in the antibiotic microplates according to an established plate plan, at a rate of 180 μl/well;
7. The plates are immediately incubated at 37° C.
At each time, a plate is taken out of the oven and is observed under a microscope. A series of images of a well for each condition tested is recorded. The plate is then read on the BRT after adding contrast liquid and magnetizing the plate for 1 minute.

### c) Bacterial Strains and Growth Conditions

P. aeruginosa CIP 104 116 and S. aureus CIP 76 25 were analyzed. The bacterial cells were resuspended in 100 mL of sterile BHI (Brain Heart Infusion medium) to an optical density at 600 nm OD600=0.004. This corresponded to the Initial Bacterial Suspension (IBS). The same IBS was used for the BioFilm Ring Test® and for the microscopic tests. Toner was added in IBS to obtain a final concentration of 10 μl/mL. The mixture was vortexed and 96-well polystyrene microplates were inoculated with 200 μl/well of IBS. A plate was prepared for each reading incubation time. Cultures grow at 37° C. without stirring. The lower part of the wells has a 6-mm diameter and is uncoated. The plates were not touched during growth until they were used for the experiments. For S. aureus, the effect of two antibiotics was studied in order to detect the capacities of the antibiotics. The two antibiotics tested were Vancomycin and fusidic acid at a respective concentration of 4 μg/mL and 2 μg/mL. In these cases, the wells were inoculated after being loaded with 20 μl of antibiotics at the aforementioned concentrations. These two antibiotics are known to have different mechanisms of action. Vancomycin acts on the bacterial wall while fusidic acid alters protein synthesis.

### d) Microscopy and Image Processing

The adhesion kinetics were analyzed every 5 minutes for P. aeruginosa and every 10 minutes for S. aureus until the immobilization of the beads. For each reading incubation time, a plate was taken out of the incubator and placed on an Olympus CKX53 inverted light microscope equipped with a 40× lens. This magnification was a good compromise between the number of particles observed and plotted and the resolution of the particles. The devices used, in particular for the image capture, were a PCO Edge camera marketed by PCO or a Basler Ace camera, the camera being connected to the video output of the microscope. The first one is equipped with a CCD sensor with 4.2 million pixels and the other with a CMOS sensor with 1.3 million pixels. The difference between the two sensors has no impact on the study. The series of images were captured at a rate of 200 images per second with 0.5 ms of illumination time per image to respect the saving capacity of the computer and the frequency of measurements. Several thousand images per series were taken for each well, at a different location, near the center of the wells. The area of the well bottom taken by the image represented between 600 μm² and 1000 μm² depending on the size of the image. The displacement of the particles was confined vertically by observing the lower part of the well. Nevertheless, the particles were allowed to leave the field of view. The first microplate was read immediately, without being placed in the incubator. The tracking of the displacement of the particles was determined according to the method described below, making it possible to determine the displacement of the centers of the beads between two consecutive images. The tracking of the particles on a series of several images made it possible to determine and obtain the trajectory of each of them. The resolution of the tracking and optical tracking was rated at ±0.045 pixel.

### e) Verification of Biofilm Formation With the BioFilm Ring Test (BRT)

Verification of biofilm formation was carried out after each microscopic capture using what is called the Biofilm Ring Test® (BRT). This is the process described in Chavant et al. “A new device for rapid evaluation of biofilm formation potential by bacteria. J Microbiol Methods. 2007 Mar; 68 (3): 605-12 [29] based on the immobilization of magnetic beads inoculated in bacterial suspension using adherent cells. The strip wells were covered with contrast liquid. Then, the plate was placed for one minute on a test block made of permanent magnets placed under each center of the wells. After magnetization, the plate was digitized to obtain an image of the bottom of the plate. The adhesion capacity of each strain was expressed in the form of the biofilm index (BFI) calculated by the BioFilm Element software.

### f) Enumeration of Adhered Bacteria

An enumeration of the adherent bacteria was carried out to link the formation of the biofilm to the microbiological activity of the S. aureus strain. The effect of the antibiotics was assessed by the number of colonies that adhered during the growth of the biofilm.

### g) Analysis of Trajectories

Global analysis: a first analysis on the bead trajectories was the study of the evolution of the standard deviation of the displacement distribution of all the beads tracked for each incubation time. The observed trajectory pi consisted of Nt,i successive displacements traveled by the particles or beads during a constant time interval corresponding to the time interval between two images as pi=(xi, yi), i=1, . . . , Nt,i. Nt,i is the length of the trajectory i. The values of the displacements traveled by each bead were concatenated in a vector P such as P={p1, p2, pNb}. Nb is the number of beads for a given incubation time. P was reshaped so that it is a one-dimensional size vector

$$( 1 , {\sum }_{i = 1}^{{N}_{b}} {N}_{t , i} ) .$$

For each incubation period, an evaluation of the standard deviation of the displacement distribution (vector P) was performed and calculated according to the following formula:

$$σ = \sqrt{\frac{1}{m} {\sum }_{i = 1}^{m} {( Pi - \overline{P} )}^{2}}$$

with m=length(P) and P=mean(P). For each incubation time, an aggregate value representing the overall state of biofilm growth was available.

### h) Individual Contributions of Each Particle/Bead

As each trajectory provides information about its surroundings, the bead displacements were also individually assessed. The motion vector provided by tracking the displacements of all the beads/particles constitutes a set of data for the observation of hundreds of beads/particles throughout the duration of the observation of the biofilm growth. To describe these trajectories with a small number of values, the tracking vectors were replaced by characteristic criteria describing the displacement distribution along the movement. Each criterion combines the information given by each particle/bead displacement measured according to the aforementioned comparison method, in particular by comparisons of observations of digital images. It was possible to analyze the displacement more quantitatively using these different criteria. For each track, discriminating parameters were calculated.
These parameters are based on the statistical displacement distribution and the geometry of the entire trajectory. From the displacement as a function of time [X(t), Y(t)] of each particle/bead, each track/trajectory was associated with an n-uplet of seven parameters:

length c1 of the diagonal of the rectangle comprising said trajectory according to the following formula:

$${c}_{1} = \sqrt{\begin{matrix}{( max ( cumsum ( X ) ) - min ( cumsum ( X ) ) )}^{2} + \\ {( max ( cumsum ( Y ) ) min ( cumsum ( Y ) ) )}^{2}\end{matrix}}$$

the average speed c2 along/on the trajectory

$${c}_{2} = mean ( \sqrt{\frac{{X}^{2} + {Y}^{2}}{Δ t}} )$$

the standard deviation of the particle/bead velocity distribution at each point of the trajectory

$${c}_{3} = std ( \sqrt{\frac{{X}^{2} + {Y}^{2}}{Δ t}} )$$

the standard deviation of the distribution of the trajectories according to the transverse and vertical directions/axes (one parameter for each component)

$${c}_{4} = std ( X ) \\ {c}_{5} = std ( Y ) \qquad$$

the asymmetry of the displacement distribution along the transverse and vertical axes (one parameter for each component).

$${c}_{6} = skw ( X ) \\ {c}_{7} = skw ( Y ) . \qquad$$

These 7 parameters were used to evaluate the way in which the displacement and/or the trajectory of the beads takes place inside a bacterial suspension. They made it possible to demonstrate that this is not a normal displacement and/or trajectory. Also, they made it possible and may be used to classify trajectories into clusters in order to clearly differentiate between active displacements, random trajectories and blocked particles. In order to keep the most discriminating information among these criteria, a principal component analysis (PCA) was carried out on all the data. PCA is a well-known analytical method in other fields. It advantageously makes it possible to keep as much information as possible to describe the trajectories by keeping only a limited number of components, namely those which most influence the observed phenomenon. From seven parameters, three main components were built. The choice of three principal components was made so as to keep the components which express the maximum variance in the information. In other words, from a base of vectors defined by the physical criteria cᵢ, the problem is translated into a base of orthogonal vectors PCᵢ. The latter are built from linear combinations of the criteria cᵢ. The coefficients of the linear decomposition give information on the contribution of the criteria cᵢ in the description of the trajectories. However, the dimensions of the PCᵢ vectors represent abstract information, which is not relevant for the analysis. In addition, the units of the components of the vectors PCᵢ may vary from one analysis to another.
Each trajectory was then represented in the coordinate system of the three principal components (PC1, PC2, PC3). Of the three principal components above, a spatial clustering of applications based on the density of points with the DBSCAN algorithm (Density-Based Spatial Clustering of Applications with Noise) (Ester et al., 1996) was used for detecting two unsupervised clusters in the graph from the first 3 principal components. These principal elements were subsequently linked to the shape of the trajectories and, therefore, to the behavior of the beads.

### i) Reproducibility Test

Each experiment for the S. aureus strain was performed twice. In what follows, the two replicates of the assays are called Replicate 1 and Replicate 2. For the P. aeruginosa strain, the growth kinetics were carried out only once. The sensitivity of biofilm growth is very dependent on environmental conditions.

### 2. Results

S. aureus and P. aeruginosa were cultured in BHI medium in microplates at 37 ° C. in the presence of microbeads/particles. The displacement of the microbeads/particles that were moving directly over the bottom of the well was observed. These inert microbeads/particles have a spherical geometry, a homogeneous size and constitute dimensionally stable probes of the behavior of the medium surrounding the bacteria.

### 2.1. Kinetics of Biofilm Growth

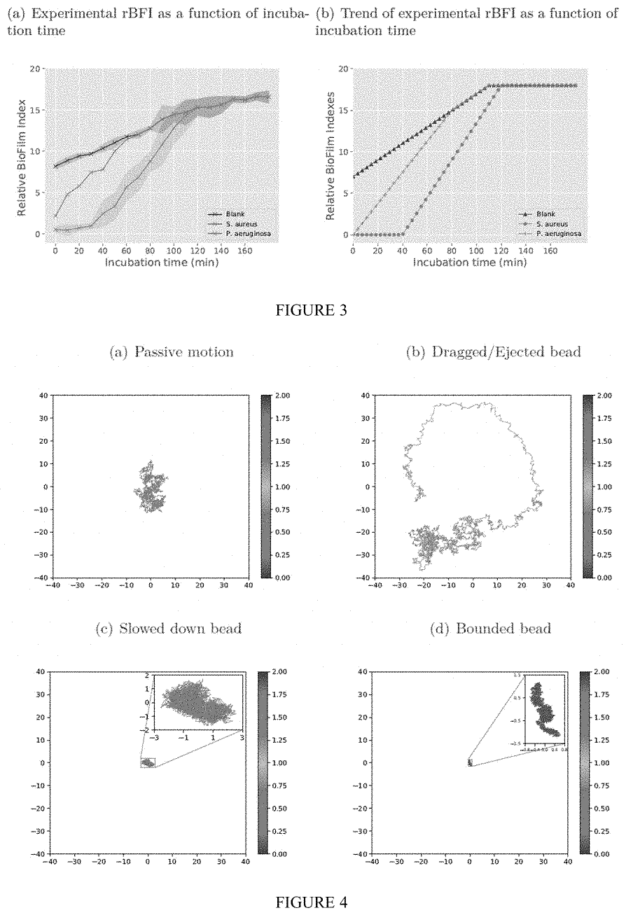

The two strains chosen have different capacities and specificities of biofilm formation as shown in FIG. 3. The kinetics of the biofilm growth deduced with the BRT for S. aureus (round-point curve) shows that the immobilization of the beads occurs at 120 minutes. In addition, the onset of blockage occurs at 40 minutes. For the P. aeruginosa strain (vertical line-dots curve), the blocking of the beads begins from the start of incubation. Complete disappearance of the spot, which signifies complete blockage of the beads, occurred at approximately 80 minutes. Qualitatively, P. aeruginosa produces a very mucous matrix in a short time compared to S. aureus which forms a biofilm over time. The rBFI for P. aeruginosa increased early on, unlike S. aureus.

### 2.2. Qualitative Analysis of Bead Displacement

Particle displacement tracking was performed by taking images of the particles/beads incorporated into the bacterial medium with high magnification, just above the bottom of the wells, individually, at short time intervals, for each incubation time. Different bead displacements could be observed in the bacterial media resulting from the different strains. The first bacterial suspension was made with a P. aeruginosa strain while the second was made with an S. aureus strain. Experimental observations showed that the bead in a bacterial suspension at 37° C. was a combination of active and thermal displacements. In addition, the bead displacement differed depending on the type of bacteria in the growth medium. The bead trajectory which results from the medium comprising P. aeruginosa differed from purely Brownian displacement. Visual observations of the trajectory of the beads within the bacterial suspension during the growth of the biofilm showed that the beads tended to move in a favored direction. While the beads in the control solution (composed of beads in the same nutrient medium) underwent a characteristic Brownian displacement, the beads in a bacterial solution/medium showed an increase in diffusion in their displacements, especially at the start of incubation. For long periods, the phenomena of “increased” diffusion could be erased. For S. aureus, the beads/particles seemed less mobile. They were experiencing random displacement, possibly disturbed by interactions with other particles. As the biofilm grew, the beads slowed down to a complete blockage. Four scenarios of bead displacement were observed during biofilm growth:

purely passive displacement: beads in an almost homogeneous medium;

dragged or ejected: beads with homogeneous or partially propelled activity;

idled: beads in a viscous material;

delimited passive: beads in a more viscous material, where the thermal energy kBT is not sufficient.

These types of displacement were observed in different ways in the two strain suspensions. Table 1 and FIG. 4 illustrate the speeds of the beads with different types of displacement.

TABLE 1
Types of displacements observed for two strains

| | Displacement type | | | |
|---|---|---|---|---|
| | | Dragged or | | Linked/Delimited |
| Strain | Passive | ejected | idled | passive |
| S. aureus | X | | X | X |
| P. aeruginosa | X | X | X | X |

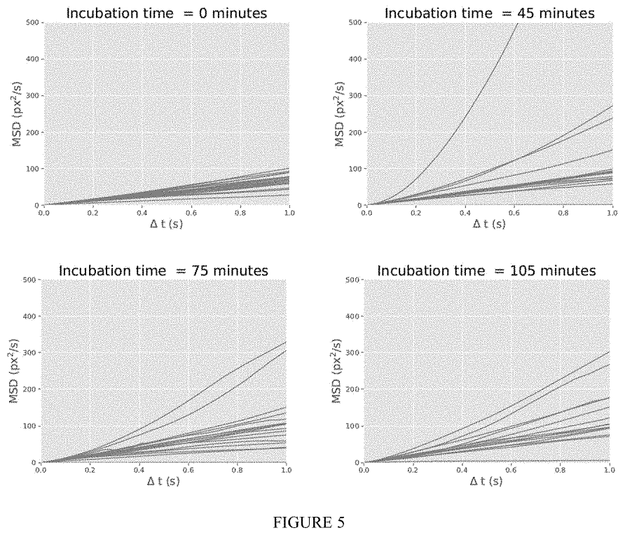

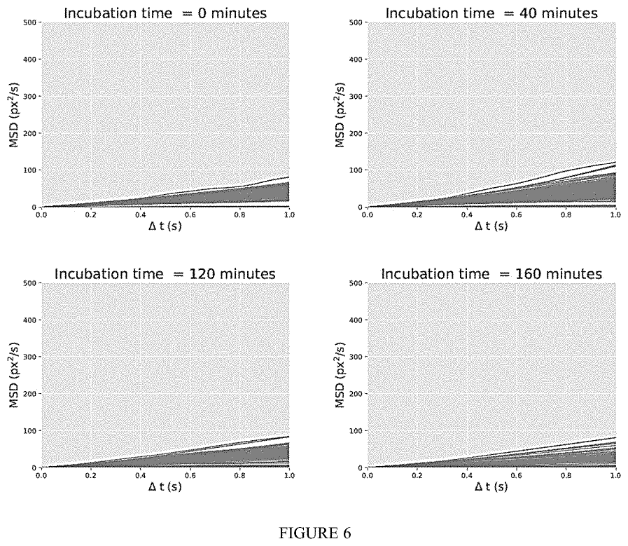

Dragged and ejected displacements appear induced by bacteria within a short time. Microorganisms like P. aeruginosa swim and sweep the surface. While inert particles undergo Brownian displacement when embedded in a liquid medium, active bacteria were able to propel themselves with a directed displacement, and thus go out of equilibrium, dragging the beads/particles. The displacement of beads/particles embedded in a solution of BHI medium and S. aureus bacteria corresponded to a random diffusion at uniform speed. There was no erratic super-diffusive displacement. As the biofilm grew, the range of displacement decreased while maintaining random characteristics. Without the action of bacteria, the passive beads had Brownian displacement. Brownian displacement is characterized by the mean squared displacement (MSD) (Δ²(Δt)) proportional to the time interval Δt between positions. Δr is the radial displacement during an interval of time Δt. The theory predicts that, for a bead with purely two-dimensional Brownian displacement, (Δr²(Δt))=4DΔt, where D is the diffusion coefficient. For a super-diffusive displacement, (Δ2Δ(t))=4Dt^{α} with α>1. In this experiment, the mean values of the squared displacement of the microbeads incorporated in the bacterial solutions were calculated during the growth of the biofilm. For each trajectory, the mean squared displacement was calculated and the MSD curves against the time interval were plotted. FIGS. 5 and 6 show the mean squared displacement versus time interval curves for the two strains during biofilm formation. For P. aeruginosa (FIG. 5), some beads show super-diffusive displacements, while for S. aureus, these behaviors were hardly observed. As shown in the Figures above, it appears that the bead displacements can provide indications of the microbiological activity of bacteria. While the bead displacement caught in an S. aureus biofilm is relatively simple, the beads in the P. aeruginosa biofilm exhibit different characteristics/behavior. This agrees with the visual observations of FIGS. 5 and 6 which show that the particle tracks consisted of straight segments of different lengths (ballistic type displacement) more or less important depending on the interactions with bacteria and the surrounding environment. The random displacement due to thermal agitation is biased by the active motility of the bacteria. The observation of bead trajectories is a step in the study of bead displacement. A quantitative analysis by defining quantitative criteria to describe the bead displacement was performed.

### 2.3 S. aureus: Quantitative Analysis of Biofilm Formation

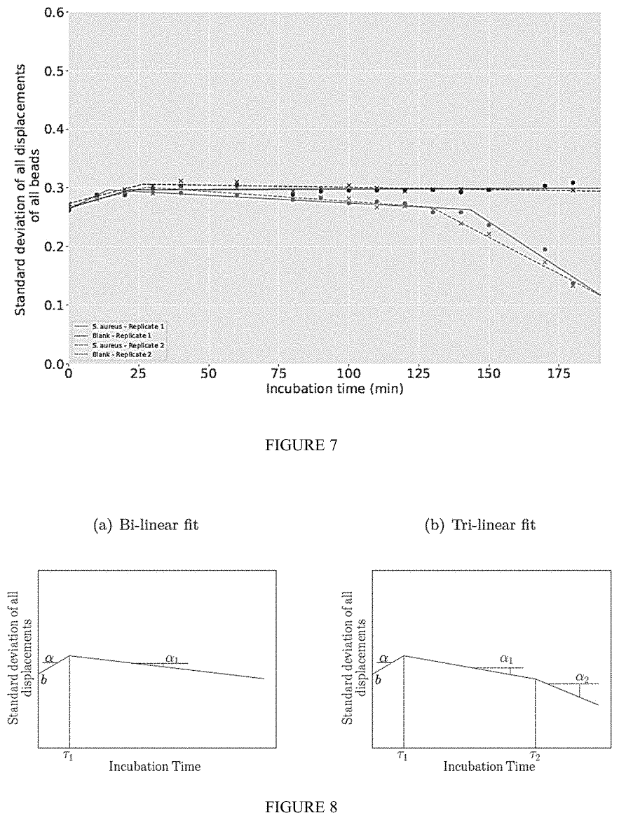

Analysis of the overall displacement distribution of all the beads tracked for each read incubation time was performed as described above. FIG. 7 represents the standard deviation of the bead displacements/trajectory distribution as a function of the incubation time for two replicates of S. aureus suspension and for the two replicates of control wells (composed of the same microbeads in BHI medium). As shown in this figure, the standard deviation increases at the start of biofilm growth. This increasing period was also observed in the control blank. Then, at 20 minutes, the standard deviation decreases for the beads in the bacterial medium while it stabilizes for the control blank. After about 140 minutes, the standard deviation of the displacements drops. The curves were approximated by piecewise linear functions. Bi-linear or tri-linear functions were used to approximate the curves as a function of the condition tested. A first approximation of the curves with a bi-linear function was carried out. If the coefficient R² was too low, a tri-linear function was chosen to adapt to the curves. The equations for the two types of fitting curves are:

$$Bi \text{-} linear regression \text{:} \{ \begin{matrix}α . t + b & for t \leq {τ}_{1} \\ {α}_{1} ( t - {τ}_{1} ) + α . {τ}_{1} + b & for {τ}_{1} \leq t \leq \end{matrix} \\ Tri \text{-} linear regression \text{:} \{ \begin{matrix}α . t + b & for t \leq {τ}_{1} \\ {α}_{1} ( t - {τ}_{1} ) + α . {τ}_{1} + b & for {τ}_{1} \leq t \leq {τ}_{2} \\ {α}_{2} ( t - {τ}_{2} ) + α {τ}_{1} + {α}_{1} ( {τ}_{2} - {τ}_{1} ) & for {τ}_{2} \leq t\end{matrix}$$

The regression made it possible to identify the analytical parameters and describes the evolution of the standard deviation with few indicators. The models and their parameters are described schematically in FIG. 8. The slopes of the second and third parts of the multilinear approximations α1 and α2, as well as the breaking times of the slopes σ1 and σ2 were reported. α1 and α2 represent a change in the behavior of the beads; σ1 and σ2 are the times of these changes. Their numerical values for the two repeats are shown in Table 2 below.

TABLE 2
Parameters deduced from the standard deviation curves in FIG. 8

| | | τ1 | τ2 | α1 | α2 |
|---|---|---|---|---|---|
| Replicate | Strain | (min) | (min) | (pixel/min) | (pixel/min) |
| 1 | S. aureus | 13.95 | 143.44 | −0.00026 | −0.0031 |
| | Blank | 21.79 | n/a | 1.63 10⁻⁵ | n/a |
| 2 | S. aureus | 27.88 | 129.44 | −0.00034 | −0.0025 |
| | Blank | 26.50 | n/a | −7.28 10⁻⁵ | n/a |

By modeling the change in standard deviation as a function of incubation time with various regular curves, two sets of conditions were distinguished.

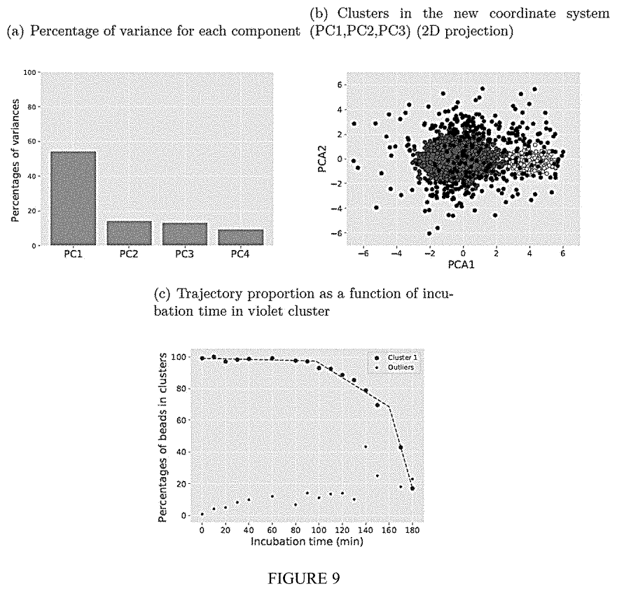

An analysis of the individual contributions of the beads was carried out in order to take into account the differences in the behavior of the beads. For each trajectory, seven criteria based on the displacement distribution were measured, as described above. Principal Component Analysis (PCA) was implemented to reduce the size of the dataset. The first principal component (PC1) represented about 60% of the total information while PC2 and PC3 each represented 14% of the information (FIG. 9). All the trajectories tracked for all the incubation times were represented in the new coordinate system consisting of (PC1, PC2, PC3).

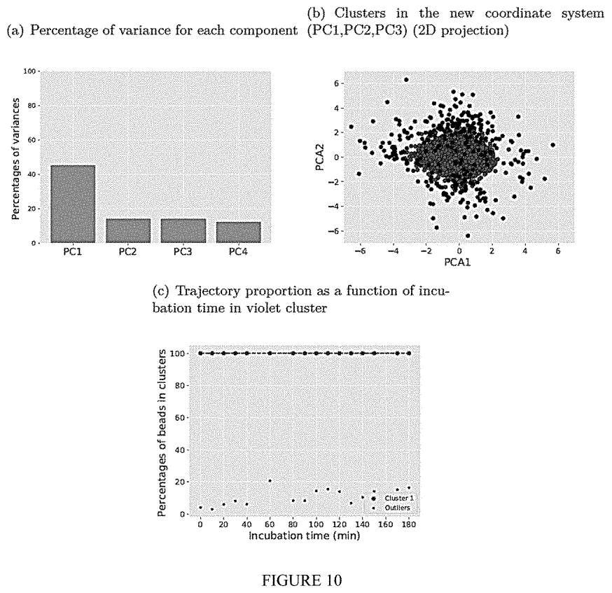

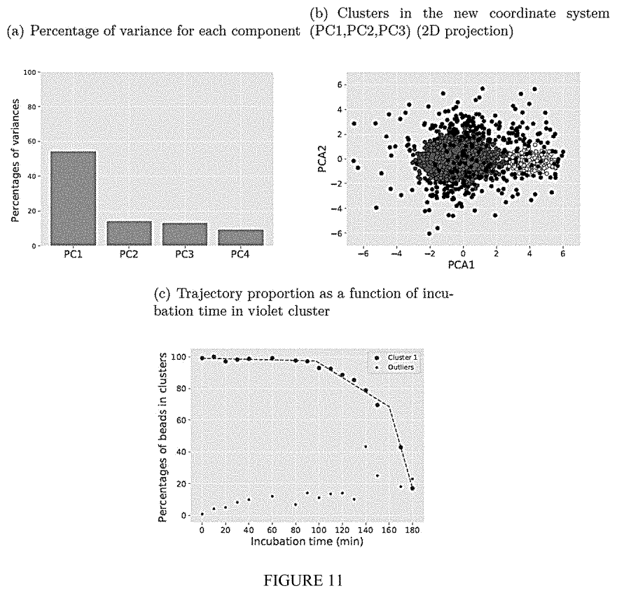

It was possible to divide all the trajectories into two groups, as shown in FIG. 9. All the beads behaved in the same way during the growth of the biofilm. The proportion of beads in each cluster as a function of incubation time was calculated and plotted (FIG. 9 (c) below). The variation of the proportion of beads in each cluster was very smooth and could be divided into three steps. The times for these changes are shown in Table 3 below. For the control well, only one group was distinguished, as shown in FIG. 10. Analysis of the second replicate (FIG. 11) demonstrated similar results.

TABLE 3
Parameters deduced from the curves of the proportions

| | | τ1 | τ2 |
|---|---|---|---|
| Replicate | Strain | (min) | (min) |
| 1 | S. aureus | 70.9 | 142.7 |
| 2 | S. aureus | 97.2 | 160 |

### 2.4. P. aeruginosa: Quantitative Analysis of Biofilm Formation

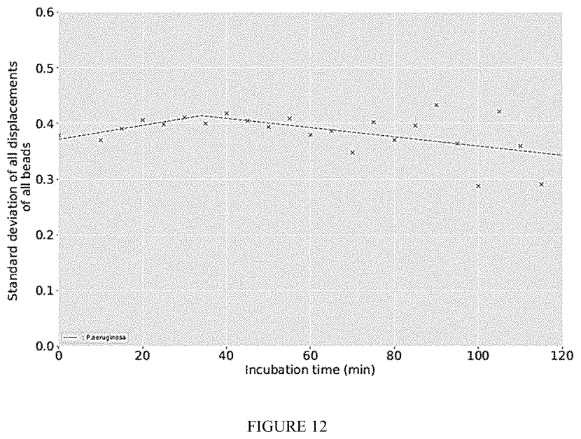

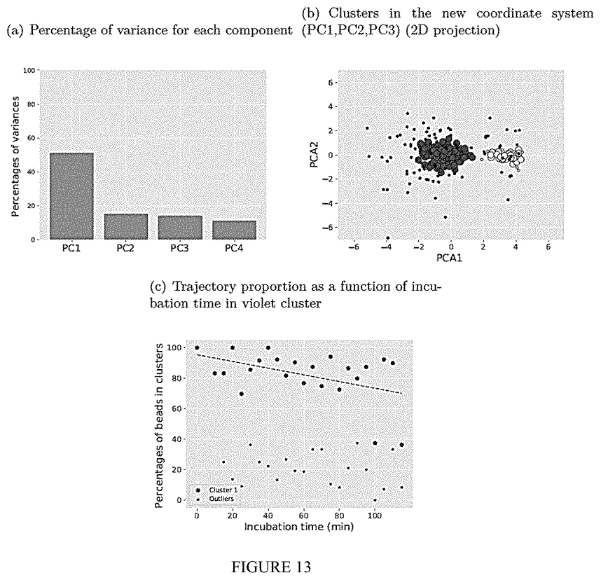

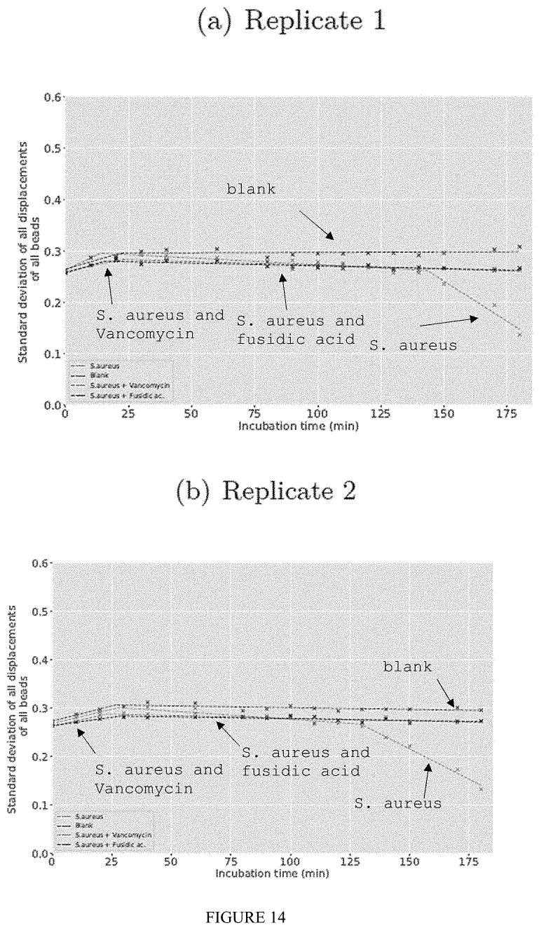

An analysis of the mass displacement distribution of all the beads tracked for each incubation time of P. aeruginosa in suspension was performed. FIG. 12 represents the standard deviation of the bead displacement distribution as a function of the incubation time. As for the suspension of S. aureus, the variation of the standard deviation was modeled by a linear function. The bi-linear model detected a change in trend at 34 minutes, where the standard deviation stops increasing. After 34 minutes, the values of the standard deviation seem to decrease overall, with a great variability in these values for the last incubation times. This variability may be due to the heterogeneity of P. aeruginosa biofilm formation. An analysis of the displacement of the individual beads was also performed. The seven parameters, previously defined, were calculated for each particle. After a PCA on all the data of the seven parameters of each trajectory, all the trajectories reported during the formation of the biofilm were in a new final coordinate system built according to three main components (PC1, PC2, PC3). As before, clusters were made to bring together the beads corresponding to the same displacement characteristics. The number of trajectories in each cluster as a function of incubation time was counted. Just like what was done for S. aureus, FIG. 13(b) below shows the distribution of the groups of points (cluster). FIG. 13(c) below shows the evolution of the number of beads belonging to a group during the growth of the biofilm for P. aeruginosa. As the biofilm grows, the general trend shows an increase in the number of blocked beads. However, the decrease is not regular. The variability of the proportion of blocked beads over the growth time shows that the number of blocked beads may vary significantly. The results on P. aeruginosa may explain that the formation of the biofilm is more heterogeneous. We find that the analysis of the individual behavior of the beads shows that, at the beginning, a particular activity in the bacterial suspension is observed and appears after the first minutes of growth.

### 2.5. S. aureus: Effect of Antibiotics on Biofilm Formation

The characterization of the effect of antibiotics is an important question in the pharmaceutical field. We tested two antibiotics here for their ability to delay or stop biofilm formation. The same processing of the experimental data is carried out. The figure below shows the evolution of the standard deviation as a function of incubation time for a suspension of S. aureus which includes antibiotics. As the biofilm grew in the presence of Vancomycin or fusidic acid, the standard deviation increased during the early periods of biofilm growth and the standard deviation increased, then decreased more slowly than without antibiotic. The bi-linear functions fit the curves of the standard deviation equation for the bacterial suspension with antibiotics. The parameters α1 and σ1 of the bi-linear approximation of the bacterial suspension with antibiotics were reported and compared with the control (blank) and the suspension of S. aureus without antibiotic in Table 4 below. The change in the standard deviation of the displacement distribution makes it possible to distinguish the presence of antibiotics in the suspension/bacterial medium. In the presence of antibiotics, the standard deviation curves resemble those of the blank control.

TABLE 4
Parameters deduced from the standard deviation curves

| | | τ₁ | τ₂ | α₁ | α₂ |
|---|---|---|---|---|---|
| Replicate | Condition | (min) | (min) | (pixel/min) | (pixel/min) |
| 1 | S. aureus | 13.95 | 143.44 | −0.00026 | −0.0031 |
| | Blank | 21.79 | n/a | 1.63 10⁻⁵ | n/a |
| | S. aureus + | 22.64 | n/a | −0.00014 | n/a |
| | Vancomycin | | | | |
| | S. aureus + | 15.44 | n/a | −0.00011 | n/a |
| | Fusidic ac. | | | | |
| 2 | S. aureus | 27.88 | 129.44 | −0.00034 | −0.0025 |
| | Blank | 26.50 | n/a | −7.28 10⁻⁵ | n/a |
| | S. aureus + | 22.93 | n/a | −0.00011 | n/a |
| | Vancomycin | | | | |
| | S. aureus + | 28.61 | n/a | −7.54 10⁻⁵ | n/a |
| | Fusidic ac. | | | | |

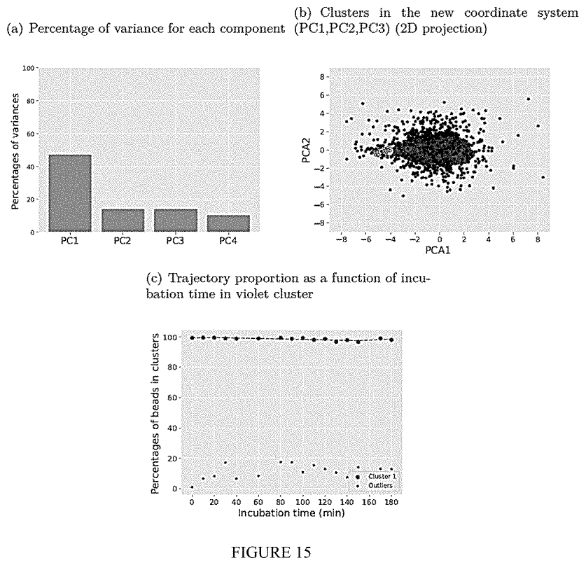

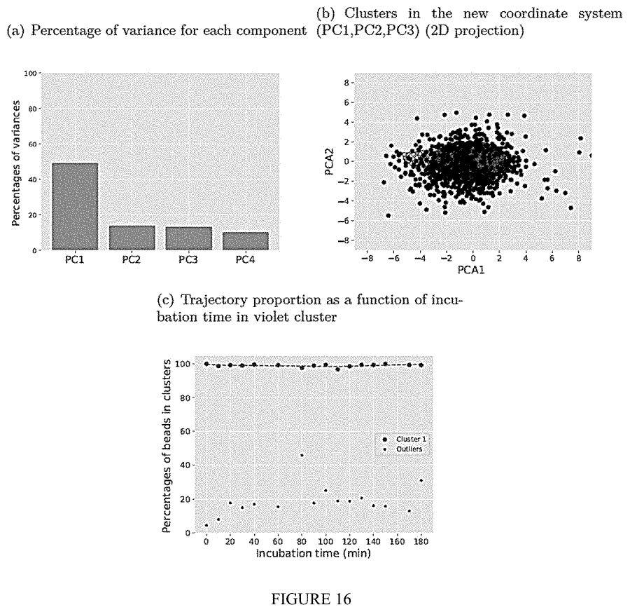

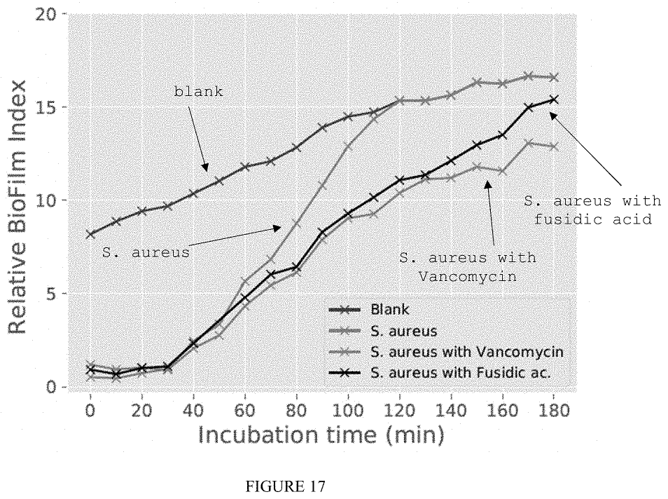

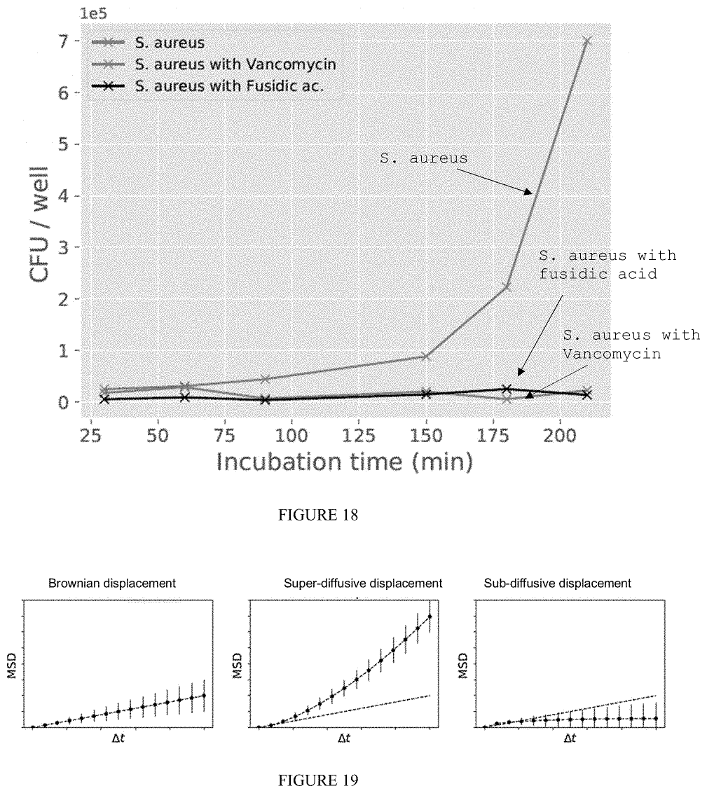

The analysis of the displacement of the individual beads after principal component analysis of the 7 parameters as mentioned above (PC1, PC2, PC3) showed that the DBSCAN algorithm makes it possible to detect a main cluster and a smaller one (FIGS. 15). The scatter plot of the points (FIGS. 15(b)) is more shifted in the direction of the first principal component than the resulting scatter plot for the control (BHI+beads only). As demonstrated, the two antibiotics prevent the immobilization of the beads, and this is clearly illustrated and demonstrated in the figures obtained, in particular after analysis of the principal components. A BRT was carried out in order to compare the results of the method with the evolution of the IAB as a function of the incubation time. For the suspension of S. aureus in the presence of antibiotics, the curves of rBFI as a function of incubation time (FIG. 17) show that the antibiotics limit the adhesion of the beads. Enumeration of adherent bacteria in wells of S. aureus with or without antibiotics (FIG. 18) demonstrated the action of both antibiotics on bacterial adhesion activity.

### 3. Discussion

Qualitative observations of bead displacement during biofilm growth of P. aeruginosa and S. aureus reveal different types of bead displacement. For both strains, the formation of a biofilm induces the formation of trapped beads. For the S. aureus strain, the range of displacement decreases with the growth of the biofilm while retaining random characteristics while the beads incorporated in the suspension of P. aeruginosa often undergo erratic ejection. The method of the invention advantageously makes it possible to detect, on a microscopic scale, biological events which may be linked to bacterial adhesion or to the growth of the biofilm. To this end, the displacement of microscopic beads incorporated in bacterial suspensions of S. aureus or P. aeruginosa was monitored using, for example, an optical device coupled to a digital image correlation algorithm. Due to the large number of data generated by the measurement of the trajectories, these were advantageously characterized by specific characteristics/parameters. On the one hand, all the displacements during the elementary time intervals of all the trajectories tracked were mixed for each reading incubation time. On the other hand, the beads were taken into account individually and the parameters/criteria determined make it possible to describe the behavior of the bead. Thanks to this tracking and analysis methodology, quantitative characteristic times during biofilm growth were determined. For the analysis of the global displacements as well as the analysis of the individual displacements of the beads, we detect a biological event at about 140 minutes of incubation for S. aureus. In addition, the classification of the beads into two groups in a well-chosen framework makes it possible to precisely detect the start of the bead blockings. For S. aureus, the onset of blocking occurs between approximately 71 minutes and 97 minutes depending on the replication involved. A second event occurs between approximately 143 and 160 minutes. These trends are consistent with the results of the BRT. No adhesion occurs in the first stages (rBFI=0), then the blocking of the beads appears progressively (increase in rBFI) until a large part of the beads is blocked (maximum rBFI). The study of the action of two antibiotics on the biofilm of S. aureus showed that the analysis of the microscopic displacements of inert beads makes it possible to distinguish the effectiveness of antibiotics compared to a bacterial suspension without antibiotics. Indeed, in the presence of antibiotics (regardless of which antibiotic), the variation in the standard deviation of displacements as a function of incubation time follows the same trend as the control. As demonstrated above, the results obtained are consistent with the BioFilm Ring Test® and the numeration, on an agar plate, of adhered bacteria. In addition, it clearly appears that the method of the invention advantageously makes it possible, and unlike the known methods, to obtain a result continuously over time thanks to its non-destructive nature. In particular, the method of the invention makes it possible to obtain a result without any action and/or addition of compounds, for example contrast agents, during bacterial culture. In addition, the method of the invention may be carried out without direct contact with the culture and thus is advantageously a non-invasive and non-destructive test while allowing numerous parameters to be obtained which may be useful in order to characterize the biofilm in real time. In addition, the method of the invention advantageously makes it possible, at the end of the incubation time of the culture, to obtain the results, to do away with the use of products/consumables, which advantageously makes it possible to reduce analysis time as well as costs, both in terms of consumables and labor. Advantageously, the method of the invention makes it possible, by tracking the displacement of the particles inserted within a biofilm, to add a temporal dimension to the measurement.

Advantageously, the method of the invention allows non-destructive detection of the formation of biofilm. In addition, the method of the invention advantageously makes it possible to detect biological events occurring during the formation of biofilm and/or distinguishing phenomena by means of the characteristics of the microscopic trajectories of inert beads. The method of the invention advantageously allows a temporal, almost continuous tracking of the kinetics of biofilm formation.

Advantageously, the method of the invention makes it possible to control the kinetics of biofilm growth, which is non-invasive and non-destructive. The method of the invention advantageously allows multiple observations of the bacterial culture during its incubation, for example, it advantageously makes it possible to multiply the observations at incubation times as close as desired, unlike known methods. Also, the method according to the invention advantageously makes it possible to obtain reliable and comparable results.

Advantageously, the method of the invention, through the use of inert microbeads within a bacterial medium and the observation of the passive displacements of said inert microbeads within a bacterial medium, makes it possible to distinguish between phenomena and characteristic times during growth kinetics of bacterial biofilms, for example P. aeruginosa and S. aureus.

Tables 5 to 13 below, summarize the parameters of the experiments and the corresponding series of images.

TABLE 5
Reference of P. aeruginosa tests

| | | | | | Number of | Min/Max | Number of |
|---|---|---|---|---|---|---|---|
| Test | Strain | Time | Antibiotic | Image size | images | traj length | beads tracked |
| P1 | P. aeruginosa | 0 | n/a | 512 × 512 | 3000 | 2704/2999 | 21 |
| P2 | P. aeruginosa | 10 | n/a | 512 × 512 | 3000 | 1147/2999 | 12 |
| P3 | P. aeruginosa | 15 | n/a | 512 × 512 | 3000 | 1005/2999 | 16 |
| P4 | P. aeruginosa | 20 | n/a | 512 × 512 | 3000 | 431/2999 | 22 |
| P5 | P. aeruginosa | 25 | n/a | 512 × 512 | 3000 | 2282/2999 | 11 |
| P6 | P. aeruginosa | 30 | n/a | 512 × 512 | 3000 | 191/2999 | 11 |
| P7 | P. aeruginosa | 35 | n/a | 512 × 512 | 3000 | 432/2999 | 16 |
| P8 | P. aeruginosa | 40 | n/a | 512 × 512 | 3000 | 237/2999 | 18 |
| P9 | P. aeruginosa | 45 | n/a | 512 × 512 | 3000 | 495/2999 | 15 |
| P10 | P. aeruginosa | 50 | n/a | 512 × 512 | 3000 | 16/2999 | 15 |
| P11 | P. aeruginosa | 55 | n/a | 512 × 512 | 3000 | 223/2999 | 26 |
| P12 | P. aeruginosa | 60 | n/a | 512 × 512 | 3000 | 151/2999 | 16 |
| P13 | P. aeruginosa | 65 | n/a | 512 × 512 | 3000 | 14/2999 | 12 |
| P14 | P. aeruginosa | 70 | n/a | 512 × 512 | 3000 | 656/2999 | 12 |
| P15 | P. aeruginosa | 75 | n/a | 512 × 512 | 3000 | 90/2999 | 19 |
| P16 | P. aeruginosa | 80 | n/a | 512 × 512 | 3000 | 659/2999 | 12 |
| P17 | P. aeruginosa | 85 | n/a | 512 × 512 | 3000 | 985/2999 | 19 |
| P18 | P. aeruginosa | 90 | n/a | 512 × 512 | 3000 | 1325/2999 | 10 |
| P19 | P. aeruginosa | 95 | n/a | 512 × 512 | 3000 | 2030/2999 | 10 |
| P20 | P. aeruginosa | 100 | n/a | 512 × 512 | 3000 | 1946/2999 | 8 |
| P21 | P. aeruginosa | 105 | n/a | 512 × 512 | 3000 | 405/2999 | 14 |
| P22 | P. aeruginosa | 110 | n/a | 512 × 512 | 3000 | 98/2999 | 16 |
| P23 | P. aeruginosa | 115 | n/a | 512 × 512 | 3000 | 2999/2999 | 12 |

TABLE 6
Tests with S. aureus, Test reference - Replicate 1 - Blank

| | | | | | Number of | Min/Max | Number of |
|---|---|---|---|---|---|---|---|
| Test | Strain | Time | Antibiotic | Image size | images | traj length | beads tracked |
| ST₀ | Control - Blank | 0 | n/a | 1000 × 1000 | 3000 | 569/2999 | 95 |
| ST₁₀ | Control - Blank | 10 | n/a | 1000 × 1000 | 3000 | 729/2999 | 193 |
| ST₂₀ | Control - Blank | 20 | n/a | 1000 × 1000 | 3000 | 56/2999 | 247 |
| ST₃₀ | Control - Blank | 30 | n/a | 1000 × 1000 | 3000 | 239/2999 | 291 |
| ST₄₀ | Control - Blank | 40 | n/a | 1000 × 1000 | 3000 | 186/2999 | 243 |
| ST₅₀ | Control - Blank | 50 | n/a | 1000 × 1000 | 3000 | 361/2999 | 277 |
| ST₆₀ | Control - Blank | 60 | n/a | 1000 × 1000 | 3000 | 204/2999 | 290 |
| ST₇₀ | Control - Blank | 70 | n/a | 1000 × 1000 | 3000 | 105/2999 | 261 |
| ST₈₀ | Control - Blank | 80 | n/a | 1000 × 1000 | 3000 | 215/2999 | 238 |
| ST₉₀ | Control - Blank | 90 | n/a | 1000 × 1000 | 3000 | 452/2999 | 215 |
| ST₁₀₀ | Control - Blank | 100 | n/a | 1000 × 1000 | 3000 | 210/2999 | 201 |
| ST₁₁₀ | Control - Blank | 110 | n/a | 1000 × 1000 | 3000 | 210/2999 | 166 |
| ST₁₂₀ | Control - Blank | 120 | n/a | 1000 × 1000 | 3000 | 171/2999 | 149 |
| ST₁₃₀ | Control - Blank | 130 | n/a | 1000 × 1000 | 3000 | 536/2999 | 147 |
| ST₁₄₀ | Control - Blank | 140 | n/a | 1000 × 1000 | 3000 | 73/2999 | 161 |
| ST₁₅₀ | Control - Blank | 150 | n/a | 1000 × 1000 | 3000 | 15/2999 | 133 |
| ST₁₆₀ | Control - Blank | 160 | n/a | 1000 × 1000 | 3000 | 259/2999 | 195 |
| ST₁₇₀ | Control - Blank | 170 | n/a | 1000 × 1000 | 3000 | 397/2999 | 131 |
| ST₁₈₀ | Control - Blank | 180 | n/a | 1000 × 1000 | 3000 | 12/2999 | 109 |

TABLE 7
Test reference - Replicate 1 - S. aureus

| | | | | | Number of | Min/Max | Number of |
|---|---|---|---|---|---|---|---|
| Test | Strain | Time | Antibiotic | Image size | images | traj length | beads tracked |
| S₀ | S. aureus | 0 | n/a | 1000 × 1000 | 3000 | 887/2999 | 118 |
| S₁₀ | S. aureus | 10 | n/a | 1000 × 1000 | 3000 | 306/2999 | 242 |
| S₂₀ | S. aureus | 20 | n/a | 1000 × 1000 | 3000 | 674/2999 | 284 |
| S₃₀ | S. aureus | 30 | n/a | 1000 × 1000 | 3000 | 112/2999 | 243 |
| S₄₀ | S. aureus | 40 | n/a | 1000 × 1000 | 3000 | 391/2999 | 255 |
| S₅₀ | S. aureus | 50 | n/a | 1000 × 1000 | 3000 | 133/2999 | 305 |
| S₆₀ | S. aureus | 60 | n/a | 1000 × 1000 | 3000 | 69/2999 | 266 |
| S₇₀ | S. aureus | 70 | n/a | 1000 × 1000 | 3000 | 174/2999 | 199 |
| S₈₀ | S. aureus | 80 | n/a | 1000 × 1000 | 3000 | 173/2999 | 222 |
| S₉₀ | S. aureus | 90 | n/a | 1000 × 1000 | 3000 | 288/2999 | 242 |
| S₁₀₀ | S. aureus | 100 | n/a | 1000 × 1000 | 3000 | 181/2999 | 207 |
| S₁₁₀ | S. aureus | 110 | n/a | 1000 × 1000 | 3000 | 160/2999 | 215 |
| S₁₂₀ | S. aureus | 120 | n/a | 1000 × 1000 | 3000 | 149/2999 | 143 |
| S₁₃₀ | S. aureus | 130 | n/a | 1000 × 1000 | 3000 | 666/2999 | 198 |
| S₁₄₀ | S. aureus | 140 | n/a | 1000 × 1000 | 3000 | 754/2999 | 66 |
| S₁₅₀ | S. aureus | 150 | n/a | 1000 × 1000 | 3000 | 174/2999 | 223 |
| S₁₆₀ | S. aureus | 160 | n/a | 1000 × 1000 | 3000 | 2392/2999 | 105 |
| S₁₇₀ | S. aureus | 170 | n/a | 1000 × 1000 | 3000 | 37/2999 | 94 |
| S₁₈₀ | S. aureus | 180 | n/a | 1000 × 1000 | 3000 | 2953/2999 | 61 |

TABLE 8
Test reference - Replicate 1 - S. aureus - Vancomycin

| | | | | | Number of | Min/Max | Number of |
|---|---|---|---|---|---|---|---|
| Test | Strain | Time | Antibiotic | Image size | images | traj length | beads tracked |
| SV₀ | S. aureus | 0 | Vancomycin | 1000 × 1000 | 3000 | 392/2999 | 169 |
| SV₁₀ | S. aureus | 10 | Vancomycin | 1000 × 1000 | 3000 | 419/2999 | 303 |
| SV₂₀ | S. aureus | 20 | Vancomycin | 1000 × 1000 | 3000 | 179/2999 | 347 |
| SV₃₀ | S. aureus | 30 | Vancomycin | 1000 × 1000 | 3000 | 314/2999 | 305 |
| SV₄₀ | S. aureus | 40 | Vancomycin | 1000 × 1000 | 3000 | 336/2999 | 243 |
| SV₅₀ | S. aureus | 50 | Vancomycin | 1000 × 1000 | 3000 | 197/2999 | 273 |
| SV₆₀ | S. aureus | 60 | Vancomycin | 1000 × 1000 | 3000 | 315/2999 | 273 |
| SV₇₀ | S. aureus | 70 | Vancomycin | 1000 × 1000 | 3000 | 420/2999 | 219 |
| SV₈₀ | S. aureus | 80 | Vancomycin | 1000 × 1000 | 3000 | 377/2999 | 236 |
| SV₉₀ | S. aureus | 90 | Vancomycin | 1000 × 1000 | 3000 | 481/2999 | 121 |
| SV₁₀₀ | S. aureus | 100 | Vancomycin | 1000 × 1000 | 3000 | 79/2999 | 226 |
| SV₁₁₀ | S. aureus | 110 | Vancomycin | 1000 × 1000 | 3000 | 356/2999 | 202 |
| SV₁₂₀ | S. aureus | 120 | Vancomycin | 1000 × 1000 | 3000 | 241/2999 | 221 |
| SV₁₃₀ | S. aureus | 130 | Vancomycin | 1000 × 1000 | 3000 | 539/2999 | 203 |
| SV₁₄₀ | S. aureus | 140 | Vancomycin | 1000 × 1000 | 3000 | 195/2999 | 160 |
| SV₁₅₀ | S. aureus | 150 | Vancomycin | 1000 × 1000 | 3000 | 30/2999 | 99 |
| SV₁₆₀ | S. aureus | 160 | Vancomycin | 1000 × 1000 | 3000 | 279/2999 | 173 |
| SV₁₇₀ | S. aureus | 170 | Vancomycin | 1000 × 1000 | 3000 | 466/2999 | 170 |
| SV₁₈₀ | S. aureus | 180 | Vancomycin | 1000 × 1000 | 3000 | 79/2999 | 123 |

TABLE 9
Test reference- Replicate 1 - S. aureus - Fusidic acid

| | | | | | Number of | Min/Max | Number of |
|---|---|---|---|---|---|---|---|
| Test | Strain | Time | Antibiotic | Image size | images | traj length | beads tracked |
| SAF₀ | S. aureus | 0 | Fusidic Acid | 1000 × 1000 | 3000 | 364/2999 | 135 |
| SAF₁₀ | S. aureus | 10 | Fusidic Acid | 1000 × 1000 | 3000 | 172/2999 | 251 |
| SAF₂₀ | S. aureus | 20 | Fusidic Acid | 1000 × 1000 | 3000 | 262/2999 | 312 |
| SAF₃₀ | S. aureus | 30 | Fusidic Acid | 1000 × 1000 | 3000 | 334/2999 | 242 |
| SAF₄₀ | S. aureus | 40 | Fusidic Acid | 1000 × 1000 | 3000 | 138/2999 | 290 |
| SAF₆₀ | S. aureus | 60 | Fusidic Acid | 1000 × 1000 | 3000 | 152/2999 | 298 |
| SAF₇₀ | S. aureus | 70 | Fusidic Acid | 1000 × 1000 | 3000 | 92/2999 | 314 |
| SAF₈₀ | S. aureus | 80 | Fusidic Acid | 1000 × 1000 | 3000 | 147/2999 | 155 |
| SAF₉₀ | S. aureus | 90 | Fusidic Acid | 1000 × 1000 | 3000 | 150/2999 | 239 |
| SAF₁₀₀ | S. aureus | 100 | Fusidic Acid | 1000 × 1000 | 3000 | 58/2999 | 236 |
| SAF₁₁₀ | S. aureus | 110 | Fusidic Acid | 1000 × 1000 | 3000 | 292/2999 | 191 |
| SAF₁₂₀ | S. aureus | 120 | Fusidic Acid | 1000 × 1000 | 3000 | 406/2999 | 171 |
| SAF₁₃₀ | S. aureus | 130 | Fusidic Acid | 1000 × 1000 | 3000 | 288/2999 | 228 |
| SAF₁₄₀ | S. aureus | 140 | Fusidic Acid | 1000 × 1000 | 3000 | 21/2999 | 181 |
| SAF₁₅₀ | S. aureus | 150 | Fusidic Acid | 1000 × 1000 | 3000 | 262/2999 | 178 |
| SAF₁₆₀ | S. aureus | 160 | Fusidic Acid | 1000 × 1000 | 3000 | 401/2999 | 135 |
| SAF₁₇₀ | S. aureus | 170 | Fusidic Acid | 1000 × 1000 | 3000 | 151/2999 | 186 |
| SAF₁₈₀ | S. aureus | 180 | Fusidic Acid | 1000 × 1000 | 3000 | 136/2999 | 191 |

TABLE 10
Tests with S. aureus, Test reference - Replicate 2 - Blank

| | | | | | Number of | Min/Max | Number of |
|---|---|---|---|---|---|---|---|
| Test | Strain | Time | Antibiotic | Image size | images | traj length | beads tracked |
| ST2₀ | Control - Blank | 0 | n/a | 1000 × 1000 | 3000 | 1968/2999 | 62 |
| ST2₁₀ | Control - Blank | 10 | n/a | 1000 × 1000 | 3000 | 338/2999 | 266 |
| ST2₂₀ | Control - Blank | 20 | n/a | 1000 × 1000 | 3000 | 384/2999 | 321 |
| ST2₃₀ | Control - Blank | 30 | n/a | 1000 × 1000 | 3000 | 288/2999 | 258 |
| ST2₄₀ | Control - Blank | 40 | n/a | 1000 × 1000 | 3000 | 227/2999 | 263 |
| ST2₅₀ | Control - Blank | 50 | n/a | 1000 × 1000 | 3000 | 207/2999 | 246 |
| ST2₆₀ | Control - Blank | 60 | n/a | 1000 × 1000 | 3000 | 26/2999 | 278 |
| ST2₇₀ | Control - Blank | 70 | n/a | 1000 × 1000 | 3000 | 45/2999 | 242 |
| ST2₈₀ | Control - Blank | 80 | n/a | 1000 × 1000 | 3000 | 191/2999 | 144 |
| ST2₉₀ | Control - Blank | 90 | n/a | 1000 × 1000 | 3000 | 249/2999 | 218 |
| ST2₁₀₀ | Control - Blank | 100 | n/a | 1000 × 1000 | 3000 | 272/2999 | 217 |
| ST2₁₁₀ | Control - Blank | 110 | n/a | 1000 × 1000 | 3000 | 303/2999 | 204 |
| ST2₁₂₀ | Control - Blank | 120 | n/a | 1000 × 1000 | 3000 | 142/2999 | 175 |
| ST2₁₃₀ | Control - Blank | 130 | n/a | 1000 × 1000 | 3000 | 351/2999 | 140 |
| ST2₁₄₀ | Control - Blank | 140 | n/a | 1000 × 1000 | 3000 | 326/2999 | 186 |
| ST2₁₅₀ | Control - Blank | 150 | n/a | 1000 × 1000 | 3000 | 344/2999 | 169 |
| ST2₁₆₀ | Control - Blank | 160 | n/a | 1000 × 1000 | 3000 | 300/2999 | 182 |
| ST2₁₇₀ | Control - Blank | 170 | n/a | 1000 × 1000 | 3000 | 281/2999 | 160 |
| ST2₁₈₀ | Control - Blank | 180 | n/a | 1000 × 1000 | 3000 | 501/2999 | 171 |

TABLE 11
Tests with S. aureus, Test reference - Replicate 2 - Strain

| | | | | | Number of | Min/Max | Number of |
|---|---|---|---|---|---|---|---|
| Test | Strain | Time | Antibiotic | Image size | images | traj length | beads tracked |
| S2₀ | S. aureus | 0 | n/a | 1000 × 1000 | 3000 | 328/2999 | 129 |
| S2₁₀ | S. aureus | 10 | n/a | 1000 × 1000 | 3000 | 75/2999 | 331 |
| S2₂₀ | S. aureus | 20 | n/a | 1000 × 1000 | 3000 | 476/2999 | 297 |
| S2₃₀ | S. aureus | 30 | n/a | 1000 × 1000 | 3000 | 265/2999 | 333 |
| S2₄₀ | S. aureus | 40 | n/a | 1000 × 1000 | 3000 | 470/2999 | 272 |
| S2₅₀ | S. aureus | 50 | n/a | 1000 × 1000 | 3000 | 88/2999 | 229 |
| S2₆₀ | S. aureus | 60 | n/a | 1000 × 1000 | 3000 | 480/2999 | 221 |
| S2₇₀ | S. aureus | 70 | n/a | 1000 × 1000 | 3000 | 59/2999 | 215 |
| S2₈₀ | S. aureus | 80 | n/a | 1000 × 1000 | 3000 | 402/2999 | 228 |
| S2₉₀ | S. aureus | 90 | n/a | 1000 × 1000 | 3000 | 488/2999 | 188 |
| S2₁₀₀ | S. aureus | 100 | n/a | 1000 × 1000 | 3000 | 214/2999 | 169 |
| S2₁₁₀ | S. aureus | 110 | n/a | 1000 × 1000 | 3000 | 316/2999 | 220 |
| S2₁₂₀ | S. aureus | 120 | n/a | 1000 × 1000 | 3000 | 576/2999 | 98 |
| S2₁₃₀ | S. aureus | 130 | n/a | 1000 × 1000 | 3000 | 188/2999 | 136 |
| S2₁₄₀ | S. aureus | 140 | n/a | 1000 × 1000 | 3000 | 417/2999 | 178 |
| S2₁₅₀ | S. aureus | 150 | n/a | 1000 × 1000 | 3000 | 2139/2999 | 119 |
| S2₁₆₀ | S. aureus | 160 | n/a | 1000 × 1000 | 3000 | 1651/2999 | 123 |
| S2₁₇₀ | S. aureus | 170 | n/a | 1000 × 1000 | 3000 | 2999/2999 | 133 |
| S2₁₈₀ | S. aureus | 180 | n/a | 1000 × 1000 | 3000 | 2999/2999 | 36 |

TABLE 12
Tests with S. aureus, Test reference - Replicate 2 - S. aureus - Vancomycin

| | | | | | Number of | Min/Max | Number of |
|---|---|---|---|---|---|---|---|
| Test | Strain | Time | Antibiotic | Image size | images | traj length | beads tracked |
| SV2₀ | S. aureus | 0 | Vancomycin | 1000 × 1000 | 3000 | 385/2999 | 155 |
| SV2₁₀ | S. aureus | 10 | Vancomycin | 1000 × 1000 | 3000 | 178/2999 | 268 |
| SV2₂₀ | S. aureus | 20 | Vancomycin | 1000 × 1000 | 3000 | 100/2999 | 263 |
| SV2₃₀ | S. aureus | 30 | Vancomycin | 1000 × 1000 | 3000 | 302/2999 | 342 |
| SV2₄₀ | S. aureus | 40 | Vancomycin | 1000 × 1000 | 3000 | 226/2999 | 275 |
| SV2₅₀ | S. aureus | 50 | Vancomycin | 1000 × 1000 | 3000 | 410/2999 | 275 |
| SV2₆₀ | S. aureus | 60 | Vancomycin | 1000 × 1000 | 3000 | 25/2999 | 204 |
| SV2₇₀ | S. aureus | 70 | Vancomycin | 1000 × 1000 | 3000 | 90/2999 | 234 |
| SV2₈₀ | S. aureus | 80 | Vancomycin | 1000 × 1000 | 3000 | 125/2999 | 278 |
| SV2₉₀ | S. aureus | 90 | Vancomycin | 1000 × 1000 | 3000 | 44/2999 | 204 |
| SV2₁₀₀ | S. aureus | 100 | Vancomycin | 1000 × 1000 | 3000 | 206/2999 | 199 |
| SV2₁₁₀ | S. aureus | 110 | Vancomycin | 1000 × 1000 | 3000 | 249/2999 | 179 |
| SV2₁₂₀ | S. aureus | 120 | Vancomycin | 1000 × 1000 | 3000 | 47/2999 | 172 |
| SV2₁₃₀ | S. aureus | 130 | Vancomycin | 1000 × 1000 | 3000 | 254/2999 | 192 |
| SV2₁₄₀ | S. aureus | 140 | Vancomycin | 1000 × 1000 | 3000 | 141/2999 | 189 |
| SV2₁₅₀ | S. aureus | 150 | Vancomycin | 1000 × 1000 | 3000 | 120/2999 | 180 |
| SV2₁₆₀ | S. aureus | 160 | Vancomycin | 1000 × 1000 | 3000 | 1598/2999 | 58 |
| SV2₁₇₀ | S. aureus | 170 | Vancomycin | 1000 × 1000 | 3000 | 151/2999 | 159 |
| SV2₁₈₀ | S. aureus | 180 | Vancomycin | 1000 × 1000 | 3000 | 74/2999 | 164 |

TABLE 13
Tests with S. aureus, Test reference - Replicate 2 - S. aureus - Fusidic acid

| | | | | | Number of | Min/Max | Number of |
|---|---|---|---|---|---|---|---|
| Test | Strain | Time | Antibiotic | Image size | images | traj length | beads tracked |
| SFA2₀ | S. aureus | 0 | Fusidic Acid | 1000 × 1000 | 3000 | 552/2999 | 193 |
| SFA2₁₀ | S. aureus | 10 | Fusidic Acid | 1000 × 1000 | 3000 | 450/2999 | 288 |
| SFA2₂₀ | S. aureus | 20 | Fusidic Acid | 1000 × 1000 | 3000 | 218/2999 | 269 |
| SFA2₃₀ | S. aureus | 30 | Fusidic Acid | 1000 × 1000 | 3000 | 161/2999 | 298 |
| SFA2₄₀ | S. aureus | 40 | Fusidic Acid | 1000 × 1000 | 3000 | 133/2999 | 212 |
| SFA2₅₀ | S. aureus | 50 | Fusidic Acid | 1000 × 1000 | 3000 | 752/2999 | 143 |
| SFA2₆₀ | S. aureus | 60 | Fusidic Acid | 1000 × 1000 | 3000 | 167/2999 | 248 |
| SFA2₇₀ | S. aureus | 70 | Fusidic Acid | 1000 × 1000 | 3000 | 67/2999 | 220 |
| SFA2₈₀ | S. aureus | 80 | Fusidic Acid | 1000 × 1000 | 3000 | 91/2999 | 240 |
| SFA2₉₀ | S. aureus | 90 | Fusidic Acid | 1000 × 1000 | 3000 | 54/2999 | 115 |
| SFA2₁₀₀ | S. aureus | 100 | Fusidic Acid | 1000 × 1000 | 3000 | 204/2999 | 166 |
| SFA2₁₁₀ | S. aureus | 110 | Fusidic Acid | 1000 × 1000 | 3000 | 127/2999 | 195 |
| SFA2₁₂₀ | S. aureus | 120 | Fusidic Acid | 1000 × 1000 | 3000 | 1012/2999 | 201 |
| SFA2₁₃₀ | S. aureus | 130 | Fusidic Acid | 1000 × 1000 | 3000 | 665/2999 | 180 |
| SFA2₁₄₀ | S. aureus | 140 | Fusidic Acid | 1000 × 1000 | 3000 | 272/2999 | 161 |
| SFA2₁₅₀ | S. aureus | 150 | Fusidic Acid | 1000 × 1000 | 3000 | 439/2999 | 184 |
| SFA2₁₆₀ | S. aureus | 160 | Fusidic Acid | 1000 × 1000 | 3000 | 112/2999 | 167 |
| SFA2₁₇₀ | S. aureus | 170 | Fusidic Acid | 1000 × 1000 | 3000 | 337/2999 | 137 |
| SFA2₁₈₀ | S. aureus | 180 | Fusidic Acid | 1000 × 1000 | 3000 | 735/2999 | 186 |

### Example 2: Example of a Particle Tracking Method

DIC requires using digital images that may translate information about the placement of material points on the surface of the material being studied. This information may be materialized by grayscale marking. For many engineering materials and for hard bone-like living materials, the pre-application of a speckle to the surface of the samples to be tested is required. Living materials sometimes have a natural grain due to their texture, which makes the use of digital image correlation suitable. This is the case, for example, for images of biofilms taken through a microscope (Mathias and Stoodley, 2009) or for the soft tissues of the leg (Bouten, 2009).

However, the use of the natural texture of living material to obtain quantitative information on its mechanical behavior may have biases. Due to its living nature, this type of material is capable of reacting actively to the mechanical stress induced by solicitation during the test. In the present document, passive microbeads are arranged randomly within the material. The DIC may be advantageously used for the purpose of tracking particles incorporated in a heterogeneous and dynamic medium. The study medium was placed in a small volume container which allows observation under an inverted optical microscope without stress. The free displacements of the beads located directly at the bottom of the container are observed. Series of images were recorded through the video output of the inverted microscope.

Brownian displacement consists of perpetual displacement (no damping due to friction phenomena), without a privileged orientation of micrometric particles in a fluid. It is the incessant collisions of the particle with the molecules of the fluid that cause the displacement of the colloid particle. In this case, the driving force is thermal, with an energy of the order of kBT, kB being the Boltzmann constant and T the temperature, expressed in Kelvin (Wirtz, 2009). Brownian displacement is related to the probability of collision with the molecules of the solvent. On average, the particle stays still because it goes in one direction as often as in the other.

For a pure Brownian displacement, the arithmetic mean of the displacements is zero (Qian et al., 1991). What characterizes a Brownian displacement is the mean square displacement (MSD). It was demonstrated in the literature that this magnitude is dependent on the characteristics of the medium containing the microbeads. For pure Brownian diffusion, the mean square displacement of particles of a radius varied linearly as a function of the measurement time interval At of the displacement. The linearity coefficient depends on the mechanical parameters of the fluid in which the particles diffuse. The relationship between the MSD and the time interval At for a two-dimensional trajectory r(t)=(x(t), y(t)) is given by:

$$MSD ( Δ t ) = ( Δ {r ( \text{?} )}^{2} ) = 4 D Δ t \text{?} D = \frac{{k}_{B} T}{\text{?} π η a}$$

$$\text{?} \text{indicates text missing or illegible when filed}$$

D is Einstein's diffusion coefficient. It is related to the dynamic viscosity η of the solvent.

$$η = \frac{{k}_{B} T}{\text{?} π Da}$$

$$\text{?} \text{indicates text missing or illegible when filed}$$

These equations are for unbiased Brownian displacement. In addition, the shape of the curve of the MSD of the particles as a function of the displacement time interval may be characteristic of the medium in which the microbeads are immersed. For a displacement qualified as sub-diffusive, the MSD is proportional to Δt^{α} with [symbol] .
For a so-called super-diffusive displacement, the MSD is proportional to Δt^{α} with α>1. The greater the time interval, the larger the error bar may be. Indeed, the greater the time interval between two positions, the fewer terms are available to calculate the mean.
FIG. 19 represents three diagrams of MSD variation as a function of time and displacement: Brownian, super-diffusive or sub-diffusive.

### Example 3: Example of a Method for Tracking Particles in an Example of Material, Localizing a Region of Interest, Reducing the Region of Interest and Validating the Metrology of Particle Tracking

### 1. Particle Tracking

The beads/particles were observed under a light microscope using a ×40 lens (×400 magnification). These were beads/particles identical to those used in Example 1. The ×400 magnification made it possible to have a good compromise between the resolution of the information on the beads (the beads were then coded on 10 pixels (px) in diameter) and the number of beads visible in an image. Series of a few thousand images were recorded. The beads were tracked individually using a Digital Image Comparison (DIC) algorithm. The formulation of the DIC in its local version was used: the focus is on a portion of the image reduced to a square of 51×51 px² around a bead and the area is calculated in its entirety. The kinematics of the displacement remain simple and was defined on this portion of the image. It is composed of a vertical displacement component and a horizontal displacement component. For each bead, the image correlation algorithm measures the displacement in pixels between a baseline image and a subsequent image in the series, in a sequential manner. This calculation was performed along the series of images in order to reconstruct the trajectory of the bead. This DIC plot allows for fast, appropriate and known error tracking. If the algorithm shows difficulties in converging, different explanations are possible:

the bead has gone out of the image frame;

the bead has gone out of the depth of field, the distorted image is then too different from the initial image;

the bead has moved too far between the initial image and the distorted image,

the algorithm would then need a non-zero initialization.

For the first case, the coordinates of the bead make it possible to consider that the trajectory goes out of the image. For the other two cases, the calculation is initialized with a previous image in the series then the calculation resumes between this new initial image and the images which follow it.

### 2. Localizing a Region of Interest

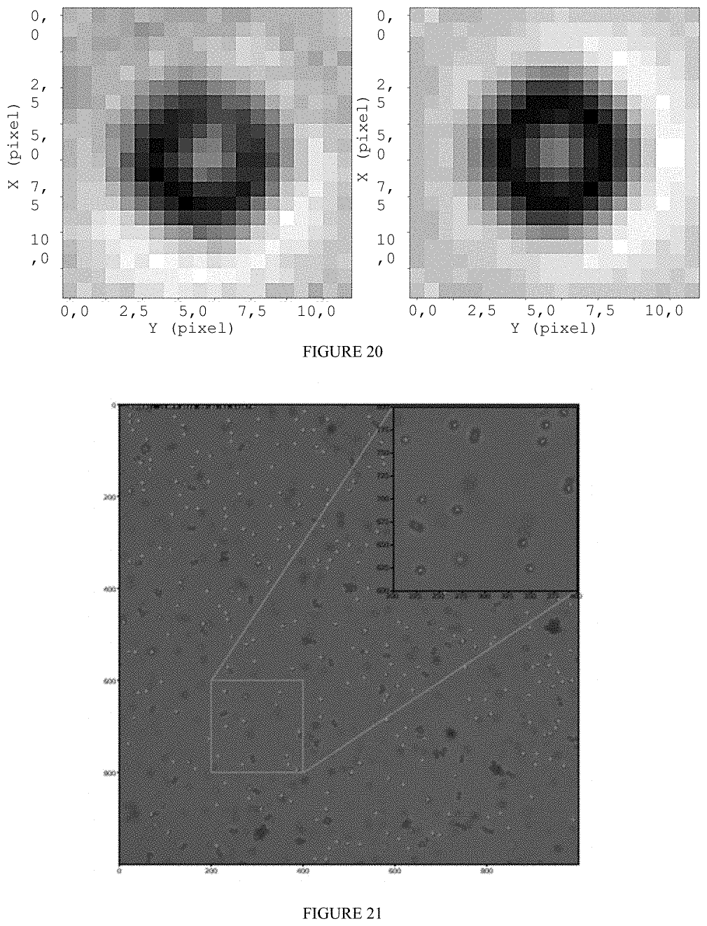

As mentioned above, bead/particle tracking was also performed individually for each particle. The objective of this part was to detect the presence of beads in the image in order to restrict the region of interest around a bead. In each series of images, a number of beads were observed ranging from a few dozen to a few hundred. It was agreed to be able to detect the beads systematically. For this, an automatic process based on image processing techniques was implemented. For each sequence of images, the first image is opened. The center of a bead in the image was manually selected by the user. A 20×20 px² Imagette_{b} image of this bead and the surrounding area was created. In order to remove the intrinsic specificity due to the choice of a particular bead, a new Imagetteᵥ image was created from the selected image. This new virtual image was composed of a linear combination of simple transformations of the base image. The new virtual image is thus created according to the following combination:

$${Imagette}_{v} = \frac{1}{4} [ {Imagette}_{b} + {Imagette}_{b}^{T} + {Imagette}_{b}^{Rh} + {Imagette}_{b}^{Rv} ]$$

where Imagette_{b} is the raw thumbnail image of the area around a bead, Imagette^{T}_{b} is the transpose of the raw thumbnail image, Imagette^{Rh}_{b} is the horizontal symmetry of the raw thumbnail image, and Imagette^{Rv}_{b} is the vertical symmetry of the raw thumbnail image. These transformations brought a correction to the symmetry of the pattern encoding the bead. FIG. 20 shows two images of a particle, the left image corresponds to the raw image and the right image to the image after transformation. Based on the OpenCV library, the area of interest localization algorithm searches for the correlation maxima between the entire image and the Imagetteᵥ image, which represents an independent bead. Correlation maxima may be present on several pixels around a bead. The center of gravity of each of the clusters formed by the set of correlation maxima around a bead was chosen to designate the center of the bead, by translating half the width and the height of the thumbnail image. This process makes it possible to detect the centers of the beads in the image. FIG. 21 corresponds to an image on which particles (black circles) are detected.

### 3. Reducing the Area of Interest

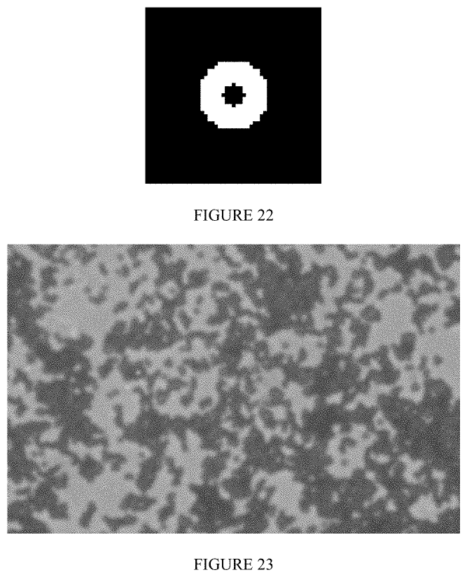

The images at the bottom of the well represent a 2D view of the three-dimensional beads. Given the sphericity of the beads, they are not coded on homogeneous gray levels. Indeed, the center of the beads was coded on lighter pixels so that the image of a bead is seen as a dark corona. In order to get rid of the lightest pixels in the center, the area of interest taken into account in the image correlation algorithm was reduced to the corona formed by the dark pixels around the edge of the bead. The bead was coded to 10 pixels (px) in diameter. On the 51×51 px² region of interest, light pixels within a 3-px radius around the center of the bead were masked. Likewise, the pixels encoding the bottom of the well beyond 13 px around the center of the bead were masked. FIG. 22 is a representation of an area of interest (particle) used to track particle displacements, in particular by image correlation. The light pixels in the center of the bead were masked because they do not provide relevant information for the DIC calculation. Likewise, the pixels at the bottom of the well beyond a small periphery around the bead were not taken into account in the calculation.

### 4. Validating Particle Tracking Metrology

If the sensor is perfect (and the light source is constant), the images of a still scene are identical. In reality, the images taken by the optical measurement system may be altered by sensor noise inherent in the electronic operation of image pickup. The noise generated by the sensor may propagate in the measurement of the displacement calculated by DIC and directly impact the measurement resolution. To calculate the sensor noise, 1000 images of a sample of beads dried on a petri dish were acquired. The focus on the dried bead aggregates was slightly shifted in depth to intentionally create a blur that smooths the grayscale gradient on the images. For each pixel pᵢ of position (xi, yi), the average of the gray levels Imi as well as the variance Ivi over the 1000 images was calculated. For a series of images of size 300×200 px², 60,000 pairs (Imi, Ivi) were calculated. They were plotted on a variance graph as a function of the average of the gray level of the pixel in question. The dispersion of the measurement points in the frame (Im, Iv) showed that, as expected, the lightest pixels exhibited the most variability. This graph profile (Im, Iv) was expected for the cameras used in field measurement. The cloud of points forms a beam oriented along an affine line. On the other hand, the bulge in the center of the beam was due to displacements of a rigid solid (Grediac and Sur). To be conservative, the standard deviation of the gray levels on the image was arbitrarily chosen as the maximum variance observed in this example. Therefore, the following formula applies:

$${σ}_{img} = \sqrt{max ( {Iv}_{i} )} = 327.31$$

The images were encoded in 16 bits. The displacement measurement resolution was then determined by the following equation (Réthoré et al., 2008; Blaysat et al., 2016):

$${σ}_{i} = {σ}_{img} \sqrt{⌈ 2 {M}^{- 1} ⌉ \text{?}} , i ∈ 1 , 2$$

$$\text{?} \text{indicates text missing or illegible when filed}$$

In practice, the values of the correlation matrix Mᵢⱼ mean that on average, the measurement resolution of the calculation by DIC for the two components of the displacement was:

$$σ \text{?} = 0.0462 px for the horizontal component of the displacement$$

$${σ}_{x} = 0.0464 px for the vertical component of the displacement$$

$$\text{?} \text{indicates text missing or illegible when filed}$$

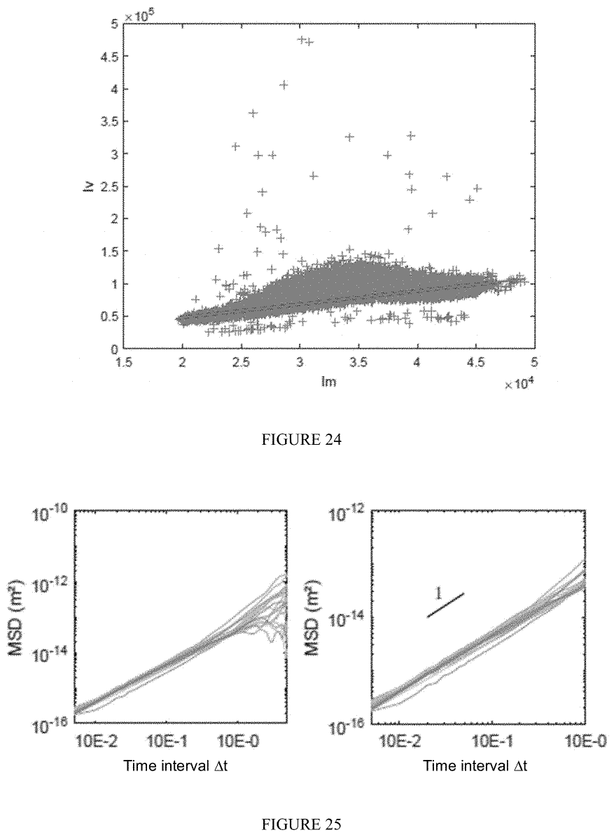

FIG. 23 corresponds to an image illustrating the background noise of the sensor. As shown, the focus has been intentionally offset to smooth grayscale gradients in the image and remove/reduce background noise. FIG. 24 is a scatter plot diagram representing the variance as a function of the average of the gray level distribution of one pixel over 1000 “identical” images.

As demonstrated, in the present example, the DIC was used for the appropriate tracking of beads/particles for which the variations and/or errors and/or inaccuracies, for example related to background noise, in terms of displacement measurement were controlled. In addition, the consideration of material heterogeneity was ensured by tracking several microbeads dispersed within the medium.

### 5. Validating in Different Types of Environments

Validation tests were carried out with standard liquids which aimed to simulate the biofilm material. Several preliminary tests of calculations of free displacement of beads moving in a homogeneous medium of known viscosity were carried out. In this example, the displacement of 1μm microbeads in standard liquids of known viscosity was studied. A first solution of glycerol at 68% (percentage by mass) was studied. For this solution, the viscosity values are tabulated (Takamura et al., 2012). Between 20° C. and 25° C., the viscosity of such a solution was approximately 16.4 mPa·s±4.8 mPa·s. The experimental value of the viscosity for this medium was calculated by calculating beforehand the diffusion coefficient D, obtained thanks to the curve MSD=f(Δt). These curves were plotted for 19 beads diffusing in the glycerol solution. On a log-log scale, the slopes of the lines are approximately equal to 1 (1.05±0.1). Therefore, the MSD values were proportional to At and the beads diffused well according to a random trend, without flow or confinement. From these curves, the viscosity values calculated for these 19 beads were on average 16.6 mPa·s±5 mPa·s. The same procedure was carried out to find the viscosity of the BHI (Brain Heart Infusion) medium used for the initial bacterial suspensions. Although the theoretical value of a BHI solution was not known, the order of magnitude of this value was 10⁻³ Pa·s. Since BHI is made from dissolving a powder in a large volume of water (37 g/L), the resulting liquid has a texture close to that of water. In a manner analogous to the approach implemented for the glycerol solution, the MSD curves as a function of At were plotted for 39 beads diffusing in a BHI solution. The displacements of the beads in this solution are of the Brownian displacement type.

The MSD curves as a function of the time interval confirmed the qualitative observations of the trajectories (FIGS. 24 and 25). Indeed, the MSD is dependent on At. The experimental viscosity measured by this test was 3.2 mPa·s±1.3 mPa·s. These first tests made it possible to confirm the measurement of a mechanical parameter under experimental conditions, for inert materials of low viscosity.

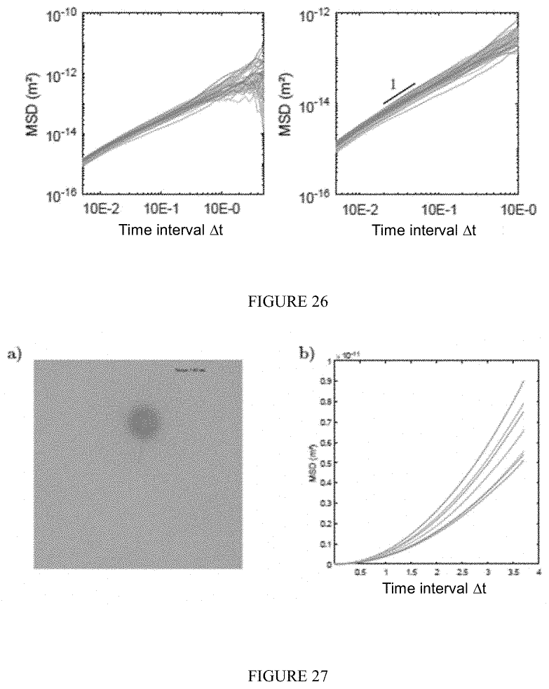

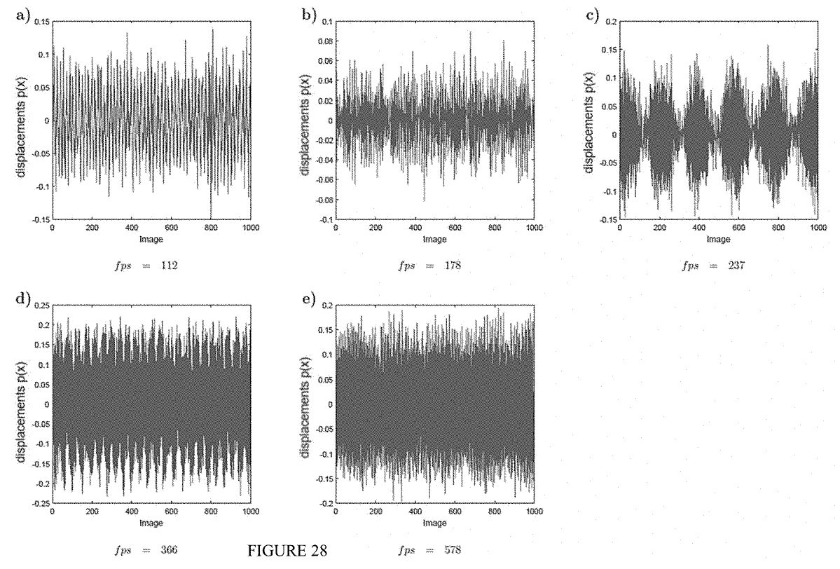

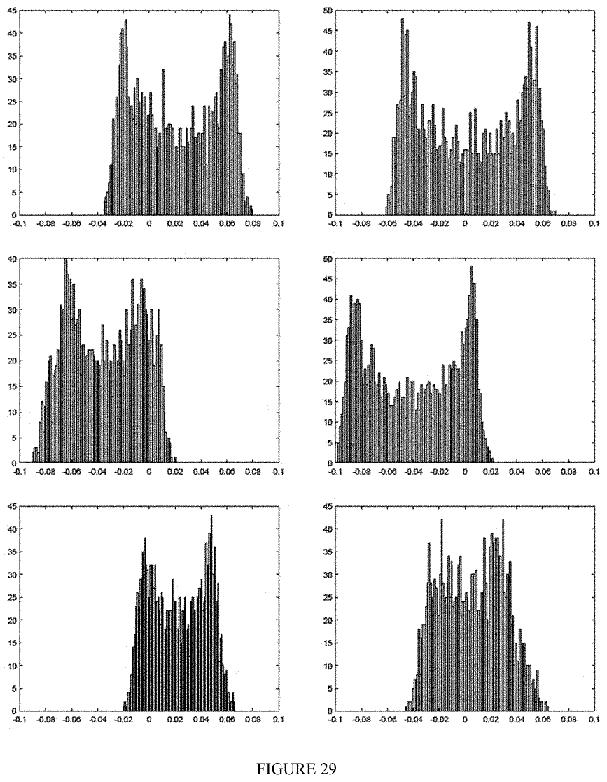

Measurements were made in media of higher viscosities and two other standard liquids one hundred times more viscous were used. The viscosities of these two samples range from approximately 500 to 700 mPa·s. The liquids used have the respective viscosity references D500 and N350 (Sigma-Aldrich) of 498.4 mPa·s and 712.9 mPa·s (values at 25° C.). Bead trajectories were extracted from series of images. Drying of the beads so as not to modify the viscosity of the liquid with the bead solution was carried out in order to avoid additional physicochemical interactions between the beads and the liquid. Observations of beads diffusing in the liquid N350 have shown bead trajectories which seem to be driven by an involuntary flow of fluid (FIG. 26). The MSD curves, as a function of the time interval considered, associated with the trajectories of six beads in this medium were typical of viscous media with the presence of flow (FIG. 27). On the other hand, for these viscosities, the average displacements of a 1-μm bead were smaller than for the liquids studied previously. The bead trajectories in the D500 liquid were displacements of very small amplitudes. Therefore, the image acquisition frequency was increased in order to capture changes in the direction of the beads, in a finer way (FIG. 29). FIG. 28 shows a component of the displacement of a bead trapped in the D500 liquid for different acquisition frequencies. The horizontal component of the bead's displacement was increased when the frequency of image acquisition was higher.

### LISTS OF REFERENCES

1. Bryers, J. (2008). Medical biofilms. Biotechnology and bioengineering, 100 (1): 1-18.
2. O'Neill, J. (2014). Antimicrobial resistance: Tackling a crisis for the health and wealth of nations.
3. Schultz, M. P., Bendick, J. A., Holm, E. R., and Hertel, W. M. (2011). Economic impact of biofouling on a naval surface ship. Biofouling, 27 (1): 87-98.
4. Costerton, J., Geesey, G., and Cheng, K. (1978). How bacteria stick. Scientific American, 238 (1): 86-95.
5. Davies, D. G. and Marques, C. N. H. (2009). A fatty acid messenger is responsible for inducing dispersion in microbial biofilms. Journal of Bacteriology, 191 (5): 1393-1403.
6. Kragh, K N, Hutchison, J B, Melaugh, G., Rodesney, C., Roberts, A E L, Irie, Y., Jensen, P. Ø., Diggle, S P, Allen, R J, Gordon, V., and Bjarnsholt, T. (2016). Role of multicellular aggregates in biofilm formation. mBio, 7 (2).
7. Flemming, H.-C., Neu, T. R., and Wozniak, D. J. (2007). The eps matrix: The “house of biofilm cells”. Journal of Bacteriology, 189 (22): 7945-7947.
8. Das, T., Sehar, S., and Manefield, M. (2013). The roles of extracellular DNA in the structural integrity of extracellular polymeric substance and bacterial biofilm development. Environmental Microbiology Reports, 5 (6): 778-786.
9. Lawrence, J. R., Korber, D. R., Hoyle, B. D., Costerton, J. W., and Caldwell, D. E. (1991). Optical sectioning of microbial biofilms. Journal of Bacteriology, 173 (20): 6558-6567.
10. Madsen, J. S., Burmolle, M., Hansen, L. H., and Sørensen, S. J. (2012). The interconnection between biofilm formation and horizontal gene transfer. FEMS Immunology & Medical Microbiology, 65 (2): 183-195.
11. Ceri, H., Olson, M. E., Stremick, C., Read, R. R., Morck, D., and Buret, A. (1999). The Calgary biofilm device: New technology for rapid determination of antibiotic susceptibilities of bacterial sbiofilms. Journal of Clinical Microbiology, 37 (6): 1771-1776
12. Harrison, J., Stremick, C., Turner, R., Allan, N., Olson, M., and Ceri, H. M. (2010). Microtiter susceptibility testing of microbes growing on peg lids: a miniaturized biofilm model for highthroughput screening. Nature Protocols, 5: 1236-1254.
13. Melchior, M., Fink-Gremmels, J., and Gaastra, W. (2007). Extended antimicrobial susceptibility assay for staphylococcus aureus isolates from bovine mastitis growing in biofilms. Veterinary Microbiology, 125 (1): 141-149.
14. Arias-Moliz, M., Ferrer-Luque, C., González-Rodriguez, M., Valderrama, M., and Baca, P. (2010). Eradication of enterococcus faecalis biofilms by cetrimide and chlorhexidine. Journal of Endodontics, 36 (1): 87-90.
15. Kharazmi, A., Giwercman, B., and Høiby, N. (1999). Robbins device in biofilm research. Methods in Enzymology, 310: 207-215.
16. Coenye, T., De Prijck, K., De Wever, B., and Nelis, H. (2008). Use of the modified robbins device to study the in vitro biofilm removal efficacy of nitradine™, a novel disinfecting formula for the maintenance of oral medical devices. Journal of Applied Microbiology, 105 (3): 733-740
17. Nickel, J., Ruseska, I., Wright, J. B., and Costerton, J. (1985). Tobramycin resistance of pseudomonas aeruginosa cells growing as a biofilm on urinary catheter material. Antimicrobial Agents and Chemotherapy, 27 (4): 619-624.
18. Goeres, D. M., Loetterle, L. R., Hamilton, M. A., Murga, R., Kirby, D. W., and Donlan, R. M. (2005). Statistical assessment of a laboratory method for growing biofilms. Microbiology,
19. Pan, B., Xie, H., and Wang, Z. (2010). Equivalence of digital image correlation criteria for pattern matching. Appl. Opt., 49 (28): 5501-5509.151 (3): 757-762.
20. Ester, M., Kriegel, H.-P., Sander, J., and Xu, X. (1996). A density-based algorithm for discovering clusters a density-based algorithm for discovering clusters in large spatial databases with noise. In Proceedings of the Second International Conference on Knowledge Discovery and Data Mining, KDD'96, pages 226-231. AAAI Press.
21. Mathias, J. D. and Stoodley, P. (2009). Applying the digital image correlation method to estimate the mechanical properties of bacterial biofilms subjected to a wall shear stress. Biofouling, 25 (8): 695-703. PMID: 20183128.
22. Bouten, L. (2009). Identification of mechanical properties of the leg's constitutive tissues for the mechanical study of the support. Theses, National School of Mines of Saint-Etienne. 181 pages.
23. Wirtz, D. (2009). Particle-tracking microrheology of living cells: Principles and applications. Annual Review of Biophysics, 38 (1): 301-326.
24. Qian, H., Sheetz, M. P., and Elson, E. L. (1991). Single particle tracking. analysis of diffusion and flow in two-dimensional systems. Biophysical Journal, 60 (4): 910-921.
25. Grediac, M. and Sur, F. 50th anniversary article: Effect of sensor noise on the resolution and spatial resolution of displacement and strain maps estimated with the grid method. Strain, 50 (1): 1-27.
26. Takamura, K., Fischer, H., and Morrow, N. R. (2012). Physical properties of aqueous glycerol solutions. Journal of Petroleum Science and Engineering, 98-99: 50-60.
27. Blaysat, B., Grédiac, M., and Sur, F. (2016). On the propagation of camera sensor noise to displacement maps obtained by dic—an experimental study. Experimental Mechanics, 56 (6): 919-944.
28. Réthoré, J., Besnard, G., Vivier, G., Hild, F., and Roux, S. (2008). Experimental investigation of localized phenomena using digital image correlation. Philosophical Magazine, 88 (28-29): 3339-3355.
29. Chavant et al “A new device for rapid evaluation of biofilm formation potential by bacteria. J Microbiol Methods. 2007 Mar; 68 (3): 605-12.

1. A method for detecting and/or tracking and/or characterizing the formation of a biofilm comprising the following steps:
a) carrying out a temporal succession of observations of a solution comprising at least one microorganism and a plurality of particles while the solution is maintained under conditions allowing the development of a biofilm by said at least one microorganism,
b) detecting the presence of the biofilm and/or characterizing the kinetics of biofilm formation on the basis of a comparative statistical analysis of the displacements of the particles observed during the various observations.

2. The method according to claim 1, wherein each observation comprises, for each particle of a set of particles observed during said observation, a determination of a trajectory corresponding to successive displacements made by said particle during said observation.

3. The method according to claim 1 comprising an overall statistical analysis of the displacements made by the particles observed during each observation and a calculation of characteristic times of the formation of the biofilm on the basis of the results of the overall statistical analysis.

4. The method according to claim 3, wherein the overall statistical analysis includes a calculation for each observation of a value of at least one statistical parameter of a displacement distribution carried out respectively by the particles of the plurality of particles and an analysis of the variations as a function of time of the values of said at least one statistical parameter obtained for the succession of observations.

5. The method according to claim 4, wherein the statistical parameter is the standard deviation of the displacement distribution made by the particles observed during said observation.

6. The method according to claim 1 further comprising a statistical analysis of the individual contributions of the particles observed from the displacements made by each particle observed during each observation and an identification of at least a percentage of particles performing the same type of displacement based on the results of statistical analysis of individual contributions.

7. The method according to claim 6, wherein the statistical analysis of the individual contributions of the particles comprises for each observation:
calculating, for each trajectory followed by a particle observed during said observation, a vector composed of parameter characteristic values of the displacements defining the trajectory concerned;
constituting a matrix from the vectors calculated for the particles observed during said observation;
decomposing said matrix by a principal component analysis;
identifying at least one major principal component among the principal components resulting from the decomposition;
generating a diagram in at least one dimension of the projections of the various vectors corresponding to the trajectories of the particles observed on said at least one predominant principal component identified.

8. The method according to claim 7, wherein said at least one characteristic parameter is selected from the group comprising:
length c1 of the rectangle diagonal comprising said trajectory according to the following formula:
$$c 1 = \sqrt{\begin{matrix}{( max ( c u m s u m ( X ) ) - min ( c u m s u m ( X ) ) )}^{2} + \\ {( max ( c u m s u m ( Y ) ) - min ( c u m s u m ( Y ) ) )}^{2}\end{matrix}}$$
where max(A) returns the largest component of vector A, min(A) returns the smallest component of vector A and cumsum(B) returns the cumulative sum of vector B.
the average speed c2 on the trajectory:
$$c 2 = mean ( \sqrt{\frac{{X}^{2} + {Y}^{2}}{Δ t}} )$$
where mean (A) returns the mean of the components of vector A
the standard deviation c3 of the speed distribution of each particle at each point of the trajectory
$$c 3 = s td ( \sqrt{\frac{{X}^{2} + {Y}^{2}}{Δ t}} )$$
where std (A) returns the standard deviation of the components of vector A
the standard deviation of the trajectory distribution along the transverse axis c4 or vertical axis c5:
$$c 4 = s t d ( X ) \\ c 5 = s td ( Y )$$
where std (A) returns the standard deviation of the components of vector A
the asymmetric distribution of trajectories along the transverse axis c6 or vertical axis c7:
$$c6 = s k w ( X )$$
$$c 7 = s kw ( Y )$$
where skw(A) returns the asymmetry coefficient of the components of vector A

9. The method according to claim 7, further comprising identifying at least one cluster of points in said diagram and calculating said percentage of particles from a percentage of points belonging to a given cluster.

10. The method according to claim 9, comprising an analysis of an evolution over time of said percentage of particles for the various observations.

11. The method according to claim 5, comprising calculating the standard deviation of the particle displacement distribution according to the following formula:
$$σ = \sqrt{\frac{1}{m} {\sum }_{i = 1}^{m} {( Pi - \overline{P} )}^{2}}$$
where P corresponds to the particle displacement vector over all observations, m=length (P), and P̅=mean(P).
